(19) United States
(12) Patent Application Publication
(10) Pub. No.: US 2002/0019543 A1
(43) Pub. Date: Feb. 14, 2002

(54) Thiol derivative, metallo-beta-lactamase inhibitors

(76) Inventors: James M. Balkovec, Martinsville, NJ (US); Mark L. Greenlee, Rahway, NJ (US); Milton L. Hammond, Somerville, NJ (US); James V. Heck, Rahway, NJ (US)

Correspondence Address: MERCK AND CO INC, P O BOX 2000, RAHWAY, NJ, 070650907

(21) Appl. No.: 09/741,644

(22) Filed: Dec. 20, 2000

Related U.S. Application Data
(62) Division of application No. 09/589,470, filed on Jun. 7, 2000, now abandoned.

(51) Int. Cl. C07D 333/76; C 07D 3 7/91; C07D 213/32

(52) U.S. Cl. 549/43; 549/461; 546/301; 562/426; 562/427; 549/499; 549/79

(57) ABSTRACT

Ar is optionally substituted with 1 to 3 R_{X} groups, and where n is 0, 1, 2 or 3; and a group of formula III:

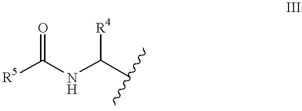

herein:

R⁴ is selected from hydrogen; and straight or branched alkyl;

R⁵ is selected from hydrogen; straight, branched, unsaturated or alicyclic alkyl, optionally substituted with 1 to 3 R_{X} groups, where the alkyl group is optionally interrupted by X, where X is selected from O, S, NH and N(COCH₃); allyloxy and 9-fluorenylmethyloxy; and (CH₂)ₙAr, where Ar is selected from phenyl, furanyl, thienyl, pyridyl, naphthyl, biphenyl, dibenzofuranyl, dibenzothienyl, fluorenyl and fluorenonyl, where n is 0, 1, 2 or 3, and where Ar is optionally substituted with 1 to 3 R_{X} groups; and

R_{X} is selected from OR, CN, C(O)NH₂, C(O)NHR, C(O)N(R)₂, OC(O)NH₂, OC(O)R, CHO, SO₂NH₂, SOR, CF₃, C(O)R, COOR, F, Cl, Br, I, OCH₂Ph, NHR, N(R)₂,, NHCOR, NHCO₂t-Bu, NHCO₂allyl, NH₂, and R, where R is hydrogen, C₁ to C₁₅ alkyl, or aryl.

The invention is further directed to a pharmaceutical composition containing the compound, as well as a method for treating bacterial infections in animals or humans, wherein the composition can be administered in combination with a β-lactam antibiotic.

### BACKGROUND OF THE INVENTION

Carbapenems, such as imipenem and meropenem, are potent broad-spectrum, β-lactam antibiotics that are widely used to treat a variety of serious infections. Among the favorable features of carbapenems are that they resist inactivation by most active-site serine β-lactamases and retain their activity against strains producing these enzymes. However, carbapenems, as well as penicillin and cephalosporin members of the B-lactam family, are efficiently hydrolyzed by the zinc-dependent molecular class B metallo-β-lactamases (MBLs). Bacteria that express MBLs show significantly reduced sensitivity to carbapenems and other β-lactam antibiotics. Consequently, MBLs present a serious threat to the clinical utility of the β-lactam class of antibiotics.

MBLs have now been identified in a number of pathogenic bacterial species including Bacillus cereus, Bacteroides fragilis, Aeromonas hydrophila, Klebsiella pneumoniae, Pseudomonas aeruginosa, Serratia marcescens, Stenotrophomonas maltophilia and Shigella flexneri. MBLs are not inactivated by currently available inhibitors of the active-site serine β-lactamases such as clavulanic acid or sulbactam. Consequently, there is a critical need for metallo-β-lactamase inhibitors that, when administered in combination with a β-lactam antibiotic, overcome MBL-mediated resistance in bacteria.

### SUMMARY OF THE INVENTION

The present invention relates to novel thiol derivative compounds, pharmaceutically acceptable salts, and biolabile esters thereof, useful for inhibiting the activity of metallo-β-lactamases and treating bacterial infections, characterized by the general formula (I):

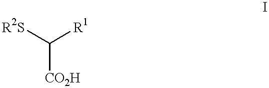

wherein:

R¹ is selected from straight, branched, unsaturated or alicyclic alkyl, optionally substituted with from 1 to 3 R_{X} groups; and (CH₂)ₙAr, where Ar is an aryl selected from the group consisting of phenyl, furanyl, thienyl, pyridyl, naphthyl, biphenyl, dibenzofuranyl, dibenzothienyl, fluorenyl and fluorenonyl, where n is 0, 1, 2 or 3, and where Ar is optionally substituted with 1 to 3 R_{X} groups;

R²is selected from hydrogen; and a group of formula II:

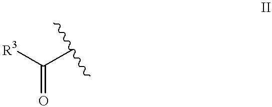

wherein:

R³ is selected from hydrogen; straight, branched, unsaturated or alicyclic alkyl, optionally substituted with from 1 to 3 R_{X} groups; (CH₂)ₙAr, where Ar is an aryl selected from phenyl, furanyl, thienyl, pyridyl, naphthyl, biphenyl dibenzofuranyl, dibenzothienyl, fluorenyl and fluorenonyl, where Ar is optionally substituted with 1 to 3 R_{X} groups, and where n is 0, 1, 2 or 3; and a group of formula III:

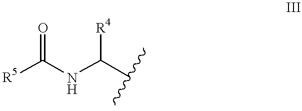

wherein:

R⁴ is selected from hydrogen; and straight or branched alkyl;

R⁵ is selected from hydrogen; straight, branched, unsaturated or alicyclic alkyl, optionally substituted with 1 to 3 R_{X} groups, where the alkyl group is optionally interrupted by X, where X is selected from O, S, NH and N(COCH₃); allyloxy and 9-fluorenylmethyloxy; and (CH₂)ₙAr, where Ar is selected from phenyl, furanyl, thienyl, pyridyl, naphthyl, biphenyl, dibenzofuranyl, dibenzothienyl, fluorenyl and fluorenonyl, where n is 0, 1, 2 or 3, and where Ar is optionally substituted with 1 to 3 R_{X} groups; and

R_{X} is selected from OR, CN, C(O)NH₂, C(O)NHR, C(O)N(R)₂, OC(O)NH₂, OC(O)R, CHO, SO₂NH₂, SOR, CF₃, C(O)R, COOR, F, Cl, Br, I, OCH₂Ph, NHR, N(R)₂, NHCOR, NHCO₂t-Bu, NHCO₂allyl, NH₂, and R, where R is hydrogen, C₁ to C₁₅ alkyl, or aryl.

The invention is further directed to a pharmaceutical composition containing the thiol derivative compound, as well as a method of treating bacterial infections in animals or humans, wherein the composition is administered in combination with a β-lactam antibiotic.

### DETAILED DESCRIPTION OF THE INVENTION

Unless otherwise specified, the term “alkyl” is defined as monovalent alkane derivatives containing from about 1 to about 15 carbon atoms, interconnected by single or multiple bonds, including straight, branched, unsaturated and alicyclic which are optionally substituted with 1 to 3 R_{X}. The term “straight alkyl” refers to C₁ to C₁₅ alkyls having one continuous chain of hydrocarbons. Examples of straight alkyl groups include, but is not limited to, methyl, ethyl, propyl, butyl, pentyl and hexyl. The term “branched alkyl” is defined as monovalent hydrocarbons have one or more non-continuous hydrocarbons linked to a main hydrocarbon chain. Examples of branched alkyl groups include, but is not limited to, isopropyl, isobutyl, t-butyl, isopentyl and neopentyl. The term “alicyclic alkyl” refers to hydrocarbon compounds which contain a saturated ring in its structure. Examples of alicyclic alkyls include, but is not limited to, cyclopropyl, cyclobutyl, cyclopentenyl, methylcyclopentyl and cyclohexyl. The term “unsaturated alkyl” refers to hydrocarbon compounds containing one or more elements of which the total valence is unsatisfied or is satisfied by union with another atom of the same element. Aryl, “Ar”, is defined as an aromatic ring substituents, including heteroaryls, having a hydrogen atom removed therefrom as well as fused ring compounds thereof. Examples of aryls include, but is not limited to, benzyl, furanyl, thienyl, pyridyl, naphthyl, biphenyl, dibenzofuranyl, dibenzothienyl, fluorenyl and fluorenonyl. Heteroatoms are independently defined as oxygen, sulfur and nitrogen atoms. Alkylcarbonyl and arylcarbonyl are defined as alkyl and aryl groups bonded to a carbonyl group, C(O).

In one preferred embodiment of the invention, stereoisomers of the thiol derivative compound, pharmaceutically acceptable salts, and biolabile esters thereof, can be utilized to effectively inhibit the activity of metallo-β-lactamases. The stereoisomers of the compound are characterized by formulae Ia and Ia′:

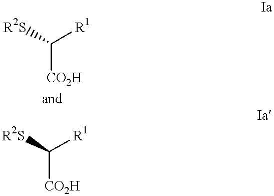

wherein

R¹, R², R³, R⁴, R⁵, R_{X} and all other variables are as originally defined.

In another preferred embodiment, where the stereoisomera are of formulae Ia and Ia′; R¹ is selected from straight, branched, unsaturated or alicyclic alkyl, optionally substituted with from 1 to 3 R_{X} groups; and (CH₂)ₙAr, where Ar is an aryl selected from the group consisting of phenyl, furanyl, thienyl, pyridyl, naphthyl, biphenyl, dibenzofuranyl, dibenzothienyl, fluorenyl and fluorenonyl, where n is 0, 1, 2 or 3, and where Ar is optionally substituted with 1 to 3 R_{X} groups; and R² is hydrogen; wherein, the thiol derivatives are characterized by the formulae:

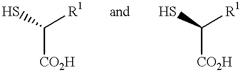

More preferably, R¹ can be selected from:

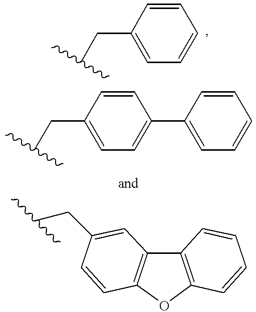

In still another preferred embodiment of the invention, where the stereoisomer of formula Ia is utilized and R² is of formula II; the thiol derivative is characterized by the formula:

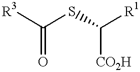

wherein:

R¹, R³, R_{X} and all other variables are as originally defined.

Within this preferred embodiment, a more preferred R¹ is (CH₂)ₙAr, where Ar is an aryl selected from biphenyl and dibenzofuranyl, where n is 1, 2 or 3, and where Ar is optionally substituted with 1 R_{X} group; and R³ is selected from methyl, and (CH₂)ₙAr, where Ar is selected from phenyl, naphthyl, pyridyl, thienyl and furanyl, where n is 0, and where Ar is optionally substituted with 1 R_{X} group. Suitable combinations of R¹ and R³ may be selected as follows:

| R¹ | R³ |
|---|---|
| 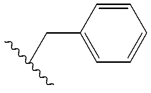 | CH₃— |
| 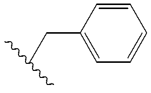 | Ph— |
| 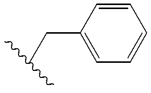 | 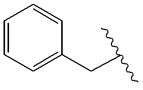 |
| 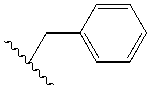 | 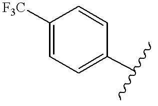 |
| 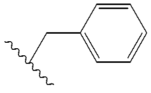 | 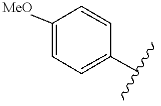 |
| 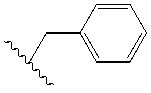 | 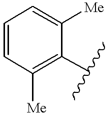 |
| 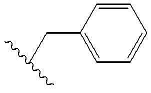 | 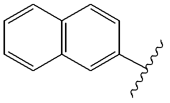 |
| 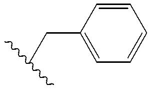 | 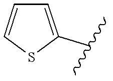 |
| 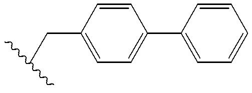 | CH₃— |
| 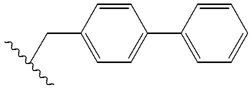 | Ph— |
| 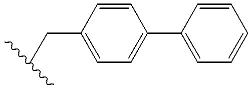 | 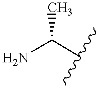 |
| 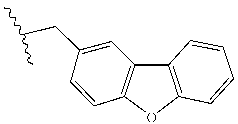 | CH₃— |
| 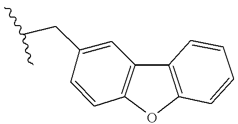 | Ph— |
| 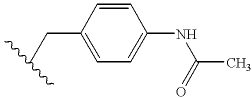 | Ph— |
| 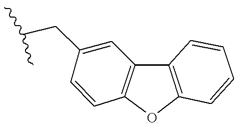 | 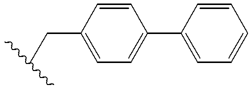 |

Another preferred embodiment of the invention, where the formual Ia′ is utilized and R² is of formula II, is characterized by the formula:

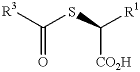

wherein R¹ and R³ combinations can be selected as follows:

| R¹ | R³ |
|---|---|
| 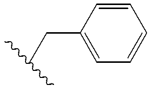 | CH₃— |
| 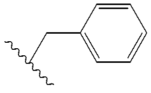 | Ph— |
| 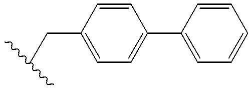 | CH₃— |
| 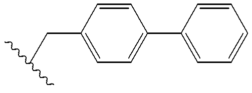 | Ph— |
| 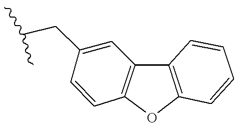 | CH₃— |
| 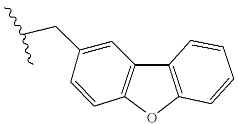 | Ph— |

Yet in another preferred embodiment of the thiol derivative, where the stereoisomer is of formula Ia, R² is of formula II, and R³ is of formula III; the compound is characterized by the formula:

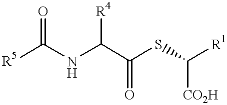

wherein R¹, R⁴, R⁵, R_{X} and all other variables are as originally defined. When R⁴ is methyl, suitable combinations of R¹ and R⁵ may be selected as follows:

| R¹ | R⁵ |
|---|---|
| 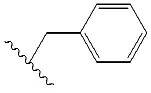 | CH₃ |
| 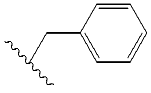 | H₂C═CHCH₂O— |
| 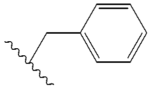 | Ph— |
| Ph— | Ph— |
| 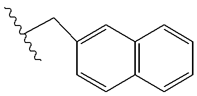 | Ph— |
| 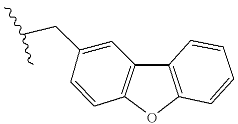 | CH₃ |
| 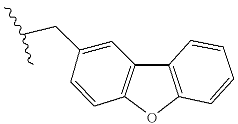 | Ph— |
| 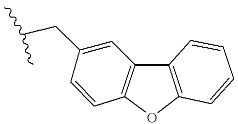 | H₂C═CHCH₂O— |
| 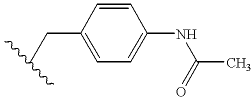 | Ph— |
| 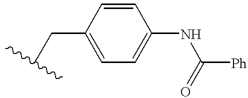 | Ph— |
| 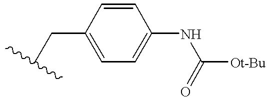 | Ph— |
| 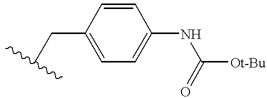 | 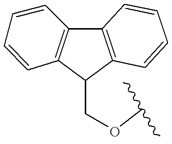 |
| 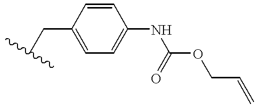 | 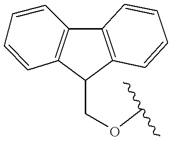 |
| 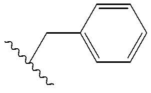 | 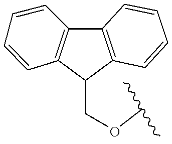 |

Within this embodiment of the invention, a more preferred R¹ is (CH₂)ₙAr, where Ar is aryl selected from biphenyl and dibenzofuranyl, where n is 1, 2 or 3; and where Ar is optionally substituted with 1 R_{X} group; and R⁴ is selected from hydrogen and methyl. Within the embodiment, when R¹ is bipehnyl, the thiol derivative is of the formula:

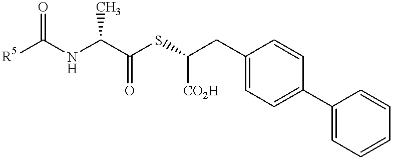

wherein R⁵ is selected from the group consisting of CH₃, CH₃CH₂, CH₃CH₂CH₂, CH₃(CH₂)₃, HO₂C(CH₂) ₂, H₂C═CHCH₂O, (CH₃)₂CHCH₂, (CH₃)₂CH, CH₃(CH₂)₄, HO₂CCH₂SCH₂, (E)—CH₃CH═CH, HO₂C(CH₂)₃, phenyl, PhOCH₂, PhCH₂, PhCH₂CH₂, (E)—PhCH═CH, PhCOCH₂CH₂, PhCONHCH₂,

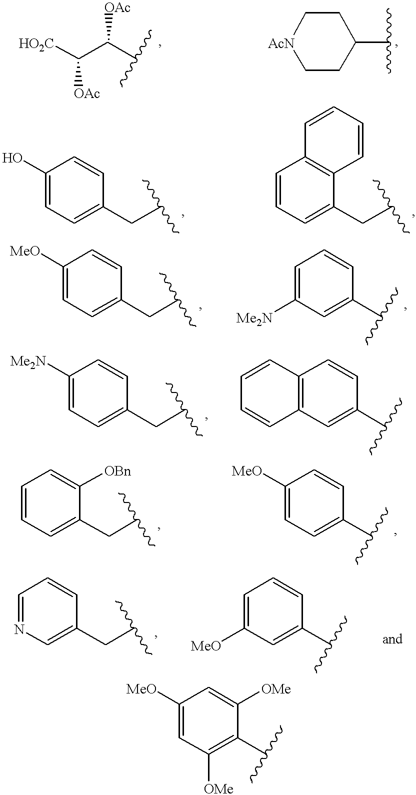

Another perferred embodiment of the invention is described by the formula:

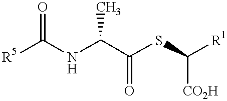

wherein R¹ and R⁵ combinations are selected from the group consisting of:

| R¹ | R⁵ |
|---|---|
| 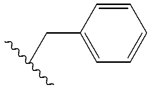 | H₂C═CHCH₂O— |
| 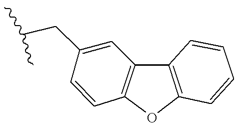 | Ph— |

Composition

The invention is further directed to a pharmaceutical composition useful for treating bacterial infections in humans and animals, wherein the composition is characterized as containing a therapeutically effective amount of the inventive thiol derivative, pharmaceutically acceptable salts, and biolabile esters thereof.

The composition can include forms for oral, topical and parenteral treatment. Suitable composition forms, include but are not limited to, tablets, capsules, lozenges, granules, powders, creams and liquid preparations, i.e. oral or parenteral solutions or suspensions.

When prepared for oral administration via capsules and tablets, the composition may contain conventional binders such as sorbitol, gelatin, syrups, acasia and other ingredients known in the art. Liquid preparations may include emulsions, syrups, elixirs and aqueous and oil suspensions.

Topical compositions may be prepared utilizing creams, lotions, powders and ointments of aqueous, alcoholic and oleaginous liquids in combination with the inventive compound, pharmaceutically acceptable salts or biolabile esters thereof.

Parenteral compositions may be prepared using the compound, salts, or esters by suspending or dissolving the derivative in a suitable carrier. For preparation purposes, the derivative may be dissolved in water for injection and filter sterilized before filling into a suitable vial or ampoule. Buffering, preservative, anesthetic agents, surfactants and wetting agents may also be dissolved in the carrier as desired.

When administered with β-lactam antibiotics, dosages of the composition that will result in a synergistic effect for treating bacterial infections in human and animals are desired as will become apparent to those skilled in the art. Generally, the composition can contain from about 0.1 to about 99.9 weight percent, based on 100 total weight percent, of the compound, pharmaceutically acceptable salts, or biolabile esters thereof. Typically, the composition can contain from about 2 to about 70 weight percent, and preferable about 20 weight percent, based on 100 total weight percent of the compound. The composition, salt or ester can contain compatible carriers known in the art, in an amounts from about 1 to about 98 weight percent, based on 100 total weight percent. Typically, the composition, salt or ester can contain carriers in an amount from about 98 to about 30 weight percent; preferably, about 80 weight percent, based on 100 total weight percent. Suitable carriers for topical application are creams, ointments and lotions having an alcohol base.

Generally, in co-administration or formulation of the compound with β-lactam antibiotics, effective dosage ratios of β-lactams may range from about 1:100 to about 100:1. The β-lactam antibiotics useful with the compound and composition of the invention include penicillins, cephalosporins and carbapenems known in the art.

Method of Treatment

The present invention is also directed to a method of treating bacterial infections in humans and animals, characterized by administering to a patient in need thereof, a therapeutically effective amount, to reduce bacterial infections, of the composition containing the thiol derivative compound.

In one preferred method of treating bacterial infections, the thiol derivative composition may be co-administered with a β-lactam antibiotic by separately administering the thiol derivative compound and the β-lactam antibiotic in close time succession, or by co-formulation, that is by preparing a single composition containing proportions of the thiol derivative compound and β-lactam antibiotic.

Suitable β-lactam antibiotics include carbapenems, penicillins, cephalosporins and other β-lactams known in the art. These compounds may also be administered in their salt and pro-drug forms.

Suitable carbapenems for co-administration with the thiol derivatives of the invention include imipenem, meropenem, biapenem, 3-[[2-(acetylamino)ethenyl]thio]-6-(1-methylethyl)-7-oxo-1-azabicyclo[3.2.0]hept-2-ene-2-carboxylic acid, 7-oxo-1-azabicyclo[3.2.0]hept-2-ene-2-carboxylic acid, and those disclosed in U.S. Pat. No. 5,478,820, incorporated herein by reference, including (1R,5S,6S,8R,2′S,4′S)-2-(2-(3-carboxyphenylcarbamoyl)pyrrolidin-4-ylthio)-6-(1-hydroxyethyl)-1-methylcarbapenem-3-carboxylic acid.

Suitable penicillins for co-administration include ampicillin, sulbenicillin, amoxycillin, propicillin, benzylpenicillin, mezlocillin, cyclacillin, phenoxymethylpenicillin, epicillin, ticarcillin, azidocillin, pirbenicillin, as well as others known in the art.

Suitable cephalosporins for co-administration include ceftriaxone, cephapirin, cephaloridine, cefazolin, cephradine, cephalexin, cephacetrile, cephaloglycin, cephalothin, cefatrizine, cefoperazone, ceftazidime, cefmetazole, cefotaxime as well as others known in the art.

Many carbapenems are susceptible to attack by a renal enzyme known as dehydropeptidase (DHP). This attack or degradation may reduce the efficacy of the carbapenem antibacterial agent. When the thiol derivative of formula I is co-administered with a carbapenem antibiotic, use of a DHP inhibitor is contemplated to be part of the present invention. Inhibitors of DHP and their use with carbapenems are disclosed in, e.g. European Patent Application Nos. 79102616.4, filed Jul. 24, 1979 (Patent No. 0007614); and 810774.3, filed Aug. 9, 1982 (Publication No. 0 072 014), both incorporated herein by reference. Typically, the method of the invention may include the co-administration suitable carbapenems, e.g. imipenem, and DHP inhibitors when desirable.

In one preferred method of the invention, the thiol derivatives may, where DHP inhibition is desired or necessary, be combined or used with the appropriate DHP inhibitor as described in the aforesaid patents and published application. The cited European Patent Applications define the procedure for determining DHP susceptibility of carbapenems and disclose suitable inhibitors, combination compositions and methods of treatment.

A preferred DHP inhibitor is 7-(L-2-amino-2-carboxy-ethylthio)-2-(2,2-dimethylcyclopropanecarboxamide)-2-heptenoic acid or a useful salt thereof.

The method of the invention is further directed to the co-administration of a serine β-lactamase inhibitor such as clavulanic acid, sulbactam or tazobactam with the thiol derivative, salt or ester to treat bacterial infections.

In yet another preferred embodiment of the invention, the thiol derivative may be co-administered with various combinations of β-lactam antibiotics, serine B-lactamase inhibitors and DHP inhibitor, as will become readily apparent to those skilled in the art.

Numerous pharmaceutically acceptable, salt-forming ions of the carboxylic acid group of the compound of formula I may be prepared according to Berge, S. M., et al. J. Pharm. Sci. 66(1): 1-16 (1977), incorporated herein by reference thereto. A preferred group of salt-forming cations are selected from aluminum, sodium, lithium, potassium, calcium, magnesium and ammonium. More preferably the cations are selected from Na⁺, Ca⁺² and K⁺. By including a suitable amount of the carbon dioxide producing compound, e.g. sodium bicarbonate or sodium carbonate, stabilized salts of the compounds may be prepared. The pharmaceutically acceptable salts referred to above also include acid addition salts. Thus, the thiol derivative compounds can be used in the form of salts derived from inorganic or organic acids. Included among such salts are the following: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate and undecanoate.

The pharmaceutically acceptable esters of the carboxylic acid group of the compounds of formula I are such as would be readily apparent to a medicinal chemist, and include, for example, those described in detail in U.S. Pat. No. 4,309,438, incorporated herein by reference. Included within such pharmaceutically acceptable esters are those which are hydrolyzed under physiological conditions, such as pivaloyloxymethyl, acetoxymethyl, phthalidyl, indanyl and methoxymethyl, and others described in detail in U.S. Pat. No. 4,479,947, incorporated herein by reference. These are also referred to as “biolabile esters”.

Biolabile esters are biologically hydrolizable, and may be suitable for oral administration, due to good absorption through the stomach or intenstinal mucosa, resistance to gastric acid degradation and other factors. Examples of biolabile ester forming moieties include acetoxymethyl, 1-acetoxyethyl, 1-acetoxypropyl, pivaloyloxymethyl, 1-isopropyloxycarbonyloxyethyl, 1-cyclohexyloxycarbonyloxyethyl, phthalidyl and (2-oxo-5-methyl-1,3-dioxolen-4-yl)methyl. These groups can be substituted in the alkyl or aryl portions thereof with acyl or halo groups.

Synthesis

Generally, the thiol derivative compound of the present invention may be synthesized in accordance with the schemes and reagents of Flow Sheets A through E, where R¹, R², R³, R⁴, R⁵and R^{X} are as previously defined, as follows:

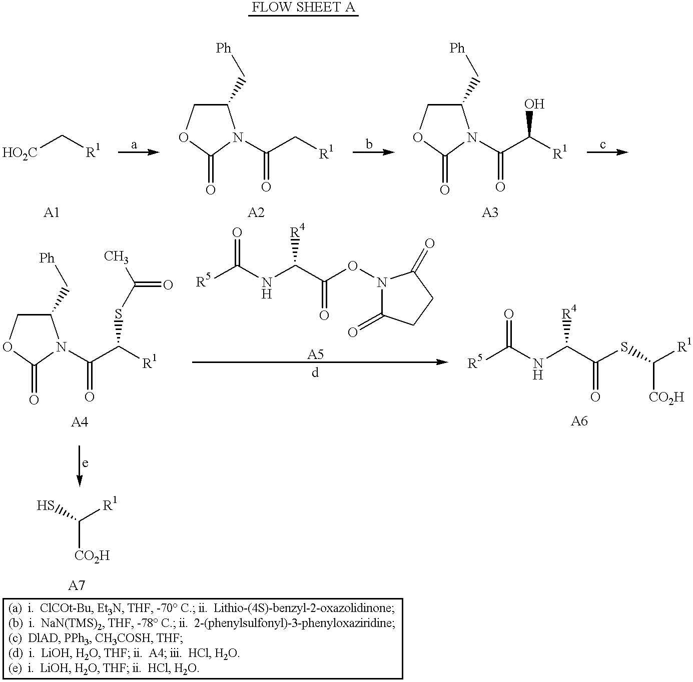

Referring to Flow Sheet A, the substituted acetic acid starting material, Al, is commercially available or can be prepared by a variety of methods known in the art. Starting material A1, wherein R¹ is previously defined, is hydroxylated on the carbon adjacent to the carboxylate group, employing a chiral auxiliary group to achieve stereoselectivity in the reaction. The hydroxyl group is then displaced with a thioacyl moiety by use of a Mitsunobu reaction. The chiral auxiliary and the acyl group on the sulfur atom are then removed by hydrolysis. The resulting thiolate is re-acylated with the desired activated acyl group to produce A6 or protonated to produce thiol A7.

Introduction of the α-hydroxy group is accomplished by an asymmetric enolate hydroxylation reaction by methods known in the art (Evans, D. A. et. al., J. Am. Chem. Soc. 1985, 107, 4346). The first step is introduction of the chiral auxiliary. A mixed anhydride is formed between the starting carboxylic acid A1 and pivalic acid by treating A1 with a tertiary amine base such as triethylamine and pivaloyl chloride in a suitable ethereal solvent such as tetrahydrofuran at reduced temperatures of from −78 to 0C. After a suitable reaction time, the resulting activated intermediate is then reacted with a solution of lithio-(4S)-benzyl-2-oxazolidinone in tetrahydrofuran at reduced temperatures of from −78 to 0C. Upon conventional isolation and purification, intermediate A2 is obtained.

Intermediate A2 is deprotonated with a strong base, e.g. sodium hexamethyldisilazide in a suitable solvent, e.g. tetrahydrofuran at reduced temperatures of from −78 to -70C. The resulting enolate is hydroxylated by addition of an appropriate oxidizing agent, e.g. 2-(phenylsulfonyl)-3-phenyloxaziridine. Upon acidification of the reaction mixture, hydroxylated compound A3 is obtained by conventional isolation and purification techniques. It will be apparent to one skilled in the art that by employing a chiral auxiliary of the opposite absolute configuration (e.g. lithio-(4R)-benzyl-2-oxazolidinone) in the first step of Flow Sheet A will make possible the synthesis of compound A3 with the alternative stereochemistry at hydroxyl group. This will make possible the synthesis of the final compounds of Flow Sheet A, A6 and A7, with the alternative stereochemistry at the sulfur-carbon bond.

Mitsunobu reaction of A3 with thioacetic acid following known procedures (Volante, R. P. Tetrahedron Lett. 1981, 22, 3119; Strijtveen, B., Kellogg, R. M. J. Org. Chem. 1986, 51, 3664) provides intermediate A4. This reaction stereoselectively introduces the sulfur atom of the compounds of the present invention. It involves reacting a dialkyl azodicarboxylate reagent, e.g. diisopropyl azodicarboxylate, with a triarylphosphine, e.g. triphenylphosphine, in a suitable solvent, e.g. tetrahydrofuran, followed by addition of A3 and thioacetic acid to the resulting reagent. The reaction is carried-out at a temperature of from about 0 to about 30C., for about 1 to about 12 hours. The product, A4, is isolated and purified by conventional methods.

Compound A6 may be synthesized from A4 by a multi-step sequence of reactions without isolation of intermediates. The first step is a hydrolysis reaction in which both the oxazolidinone chiral auxiliary and the acetyl group on the sulfur atom are removed. Aqueous lithium hydroxide is employed for this reaction along with an organic co-solvent, e.g. tetrahydrofuran. Then, without isolation, the resulting thiolate intermediate is re-acylated with an activated acylating reagent A5. After acidification, compound A6 is obtained. In Flow Sheet A, the carboxylic acid of A5 is activated as an N-hydroxysuccinimide ester. However, those skilled in the art will realize that other means of acyl activation can be employed at A5.

Compounds of structure A7 are synthesized from A4 by hydrolysis, as described above, followed by protonation of the thiolate intermediate with an acid, e.g. aqueous hydrogen chloride, to produce compound A7.

According to Flow Sheet A, the stereochemistry of the sulfur-carbon bond is partially lost due to the basic conditions of the hydrolysis reaction. Alternative syntheses of the compounds of the present invention which maintain the stereochemistry of this bond are illustrated in the following Flow Sheets.

[chemical structure]

An alternative synthesis of the compounds of the present invention is illustrated in Flow Sheet B, starting with compound A3 from Flow Sheet A. The hydroxyl group of A3 is first protected with a suitable protecting group such as allyloxycarbonyl (alloc) and then the chiral auxiliary group is removed by hydrolysis to provide compound B1. Compound B1 is attached to a solid support, making use of an acid cleavable linker group, producing B3. Removal of the alloc protecting group from the hydroxyl provides B4. Mitsunobu reaction of B4 with thioacid B5 yields thioester B6. Cleavage of the substrate from the resin under acidic conditions yields compound B7.

The solid support of Flow Sheet B is Rapp TentaGel® S—NH₂ resin which exhibits good swelling properties in organic solvents and high accessibility of its reactive sites. Other known solid supports are also suitable. To allow the desired products to be cleaved from the resin under mild conditions, attachment to the resin is made through a mild acid cleavable linker group. The linker group chosen for this purpose is the 4-(4-hydroxy-methyl-3-methoxyphenoxy)-butyrate (HMPB) group. Other known acid cleavable linker groups are also suitable. Attachment of B1 to the resin using this linker group can be accomplished by two alternative methods. In the first method, the HMPB linker group is initially derivatized as a 2,4-dichlorophenyl ester. B1 is then esterified onto the hydroxyl group of this HMPB derivative (2,4-dichlorophenyl 4-(4-hydroxy-methyl-3-methoxyphenoxy)-butyrate) to produce B2. The esterification conditions employed follow known procedures (Trost, B. M. et. al. J. Am. Chem. Soc. 1986, 51, 2370) and consist of first activating B1 with the reagent prepared from N,N-dimethylformamide and oxalyl chloride in dichloromethane solvent followed by reacting this activated intermediate with 2,4-dichlorophenyl 4-(4-hydroxy-methyl-3-methoxyphenoxy)-butyrate and pyridine to produce B2; other known esterification methods may be employed. Compound B2 is then reacted with Rapp TentaGel S—NH2 resin in the presence of 1-hydroxy-benzotriazole and N,N-diisopropylethylamine in N,N-dimethyl-formamide as solvent to produce B3. In an alternative method of attachment of B1 to the solid support, the HMPB linker group is first attached to the Rapp TentaGel S—NH₂ resin using 1-(3-dimethylamino-propyl)-3-ethylcarbodiimide hydrochloride in DMF. Compound B1 is then esterified onto this linker-resin combination (TentaGel-HMPB resin) using 1,3-diisopropylcarbodiimide and N,N-dimethylamino-pyridine in N,N-dimethylformamide as solvent to provide B3.

Removal of the alloc protecting group of B3 is accomplished by a palladium(0) catalyzed de-allylation reaction, using N-methyl-morpholine-acetic acid as the allyl acceptor and tetrakis(triphenyl-phosphine)palladium(0) as the palladium catalyst in N-methylpyrrolidinone as the solvent.

Mitsunobu reaction of B4 with a thioacid B5 yields thioester B6. Thioacids B5 can be prepared by known methods, (e.g. Yamashiro, D.; Li, C. H. Int. J. Peptide Protein Res. 1988, 31, 322. Blake, J.; Yamashiro, D. Int. J. Peptide Protein Res. 1981, 18, 383). The reaction of B4 with B5 is similar to the Mitsunobu reaction described in Flow Sheet A, except in this case B4 is bound to a solid support. In this reaction use of tris(4-chlorophenyl)-phosphine in place of triphenylphosphine is preferred. Also, the addition of an amine base such as N,N-diisopropylethylamine is beneficial. The reaction is carried-out in tetrahydrofuran as solvent and employs diisopropyl azodicarboxylate as the dialkyl azodicarboxylate reagent. Since B4 is bound to a solid support, a large excess of reagents can be used in this reaction to make it more efficient. At the end of the reaction, the excess reagents can be removed by washing the resin with appropriate solvents, e.g. N,N-dimethylformamide, tetrahydrofuran, methanol and dichloromethane.

Cleavage of final compound B7 from the solid support is accomplished with trifluoroacetic acid in dichloromethane (5% v/v). Exposure of B6 to 5% trifluoroacetic acid in dichloromethane followed by evaporation of the solution yields compound B7.

[chemical structure]

Flow Sheet C describes a further extension of the synthesis shown in Flow Sheet B, starting with compound B4. Mitsunobu reaction of B4 is carried-out using alloc-D-thioalanine dicyclohexylamine salt to provide thioester C1. This Mitsunobu reaction is analogous to that described in Flow Sheet B, except that addition of an amine base is usually not necessary since the thioacid used is already an amine salt. Next, compound C1 is reacted with anhydride C2 to produce C3 in a “trans-acylation” reaction. Similar reactions have been shown (e.g. Dessolin, M.; Guillerez, M.-G.; Thieriet, N.; Guibe, F.; Loffet, A. Tetrahedron Lett. 1995, 36, 5741, and Thieriet, N.; Alsina, J.; Giralt, E.; Guibe, F.; Albericio, F. Tetrahedron Lett. 1997, 38, 7275.). This reaction involves palladium(0) catalyzed reductive de-allylation of the alloc protected compound C1 using tetrakis(triphenylphosphine)-palladium(0) as the palladium catalyst and phenylsilane as the reducing agent in dichloromethane as solvent. The resulting deprotected amine is reacylated in situ with anhydride C2 to yield compound C3. Anhydride C2 can be pre-formed, or it can be prepared in situ by reacting two equivalents of the corresponding carboxylic acid (R⁵CO₂H) with one equivalent of N-t-butyl-N′-ethylcarbodiimide in dichloromethane. Other acylating agents can also be employed, although the use of anhydride C2 is preferred.

Exposure of C3 to 5% trifluoroacetic acid in dichloromethane followed by evaporation of the solution yields compound C4.

[chemical structure]

Flow Sheet D describes another synthesis of compounds of the present invention, starting with B4. Mitsunobu reaction of B4 is conducted with thioacid Dl to provide thioester D2. This Mitsunobu reaction is performed under conditions analogous to those described in Flow Sheet B for the reaction between B4 and B5. Compound D3 is obtained by exposure of D2 to 5% trifluoroacetic acid in dichloromethane followed by evaporation of the solution.

Compound D3 may be converted to compound D4 by cleavage of the thioacyl group. This is accomplished by reacting D3 with aqueous ammonium hydroxide in a suitable organic solvent, e.g. tetrahydrofuran in the presence of dithiothreitol, which inhibits the oxidation of thiol D4 to the corresponding disulfide. This reaction is preferably carried-out when the R³ group, previously defined, of D3 is methyl.

Flow Sheet D also illustrates the inversion of the stereochemistry of the hydroxyl group of B4 to provide D5. This is accomplished by a Mitsunobu reaction of B4 with formic acid followed by cleavage of the resulting formate ester to yield D5. This Mitsunobu reaction is similar to those described above, except that formic acid, a carboxylic acid, is employed instead of a thioacid. In this reaction, triphenylphosphine is used as the triarylphosphine reagent, and no amine base is added to the reaction. Cleavage of the formate ester to produce D5 is accomplished by reacting the product of the Mitsunobu reaction with N,N-diisopropyl-ethylamine and hydroxylamine hydrochloride employing a suitable solvent mixture, e.g. tetrahydrofuran and N,N-dimethylformamide.

Beginning with the inverted hydroxyl compound D5, Flow Sheet D operates as described above for B4, to provide compounds D7 and D8.

[chemical structure]

Flow Sheet E describes a further synthesis of compounds of the present invention. Starting with B4, Mitsunobu reaction with thioacetic acid yields E1. Cleavage of the acetyl group from the sulfur atom of El followed by reacylation with carboxylic acid E3 produces E4. Cleavage from the solid support provides compound E5.

The Mitsunobu reaction of B4 to produce E1 is carried-out in the same manner as described in Flow Sheet B for the reaction of B4 with B5 and in Flow Sheet D for the reaction of B4 with D1. Cleavage of the acetyl group of E1 is accomplished by reacting E1 with N,N-diisopropylethylamine and hydroxylamine hydrochloride employing a suitable solvent mixture such as tetrahydrofuran and N,N-dimethylformamide. The resulting thiol compound E2 is reacylated with the carboxylic acid E3 employing 1-hydroxy-7-azabenzotriazole, 1,3-diisopropylcarbodiimide and N,N-diisopropylethylamine as activating agents in N,N-dimethylformamide as solvent. Those skilled in the art will recognize that other activating agents can be used for this reaction and that activated forms of the carboxylic acid E3 (e.g. acid chloride) can also be employed for this acylation reaction. Final compound E5 is obtained by exposing E4 to 5% trifluoroacetic acid in dichloromethane followed by evaporation of the solution.

Preparations and Examples

The following preparations and examples are for illustrative purposes and are not to be construed as limiting the invention disclosed herein.

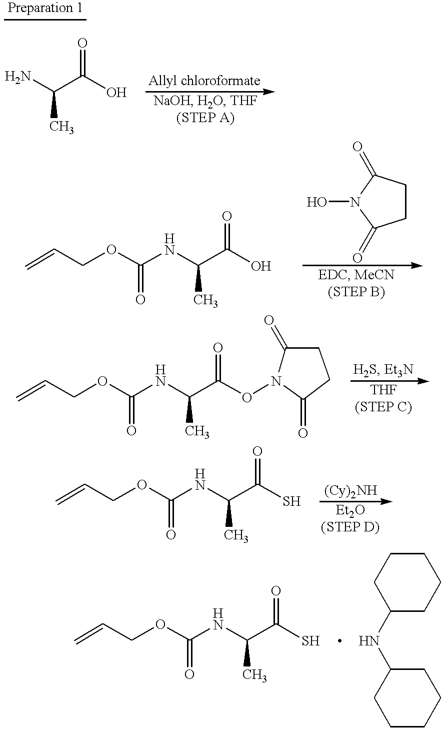

Alloc-D-thioalanine dicyclohexylamine salt

Step A

A solution of D-alanine (4.03 g, 45.2 mmol) in 100 mL of THF and 80 mL of water is cooled to 0° C. and the pH is adjusted to 9.5 by addition of 2.5 N aqueous NaOH. Neat allyl chloroformate (5.8 mL, 54 mmol) is added dropwise during about 15 min, and the pH is maintained at from about 7 to about 9 by portionwise addition of 2.5 N aqueous NaOH. After 1.5 hour, the cooling bath is removed, and most of the THF is removed by rotary evaporation. The aqueous residue is extracted twice with Et₂O and cooled to 0° C. and acidified to about pH 2.5 by addition of 12 N aqueous HCl. The resulting aqueous mixture is extracted with CHCl₃ and the combined extracts are dried over Na₂SO₄ and evaporated in vacuo to yield about 5.85 g of a colorless oil.

¹H-NMR (500 Mz, CDCl₃): δ 1.49 (d, J=7.1 Hz, 3H), 4.35-4.45 (m, 1H) 4.55-4.65 (m, 2H), 5.2-5.4 (m, 2H), 5.85-5.95 (m, 1H), 10.0-10.6 (bs, 1H).

Step B

The alloc-D-alanine product of Step A (5.85 g, 33.8 mmol) is dissolved in 70 mL of MeCN and N-hydroxysuccinimide (4.67 g, 40.6 mmol) is added thereto. The resulting solution is cooled to 0° C. and 1-(3-dimethylaminopropyl)-3-ethylcarbodiimide hydrochloride (7.78 g, 40.6 mmol) is added. Upon stirring for 4 hours, the reaction mixture is diluted with EtOAc and washed with water, sat. aqueous NaHCO₃, sat. aqueous NH₄Cl and brine. The organic layer is dried over Na₂SO₄ and evaporated in vacuo to yield a semi-solid. Recrystallization from isopropanol yields about 5.59 g of a white crystalline solid.

¹H-NMR (500 Mz, CDCl₃): δ 1.62 (d, J=7.4 Hz, 3H), 2.86 (bs, 4H), 4.55-4.65 (m, 2H) 4.70-4.85 (m, 1H), 5.2-5.4 (m, 2H), 5.85-5.95 (m, 1H).

Step C

A solution of triethylamine (1.25 mL, 8.97 mmol) in 20 mL of THF was cooled to 0° C. and hydrogen sulfide was bubbled though for 20 min. The resulting yellow solution was added via cannula during 10 min to a solution of the alloc-D-Ala-OSu product of Step B (1.613 g, 5.97 mmol) in 10 mL of THF cooled to 0° C. After 40 min, the reaction mixture was acidified with 1 N HCl. The cooling bath was removed and nitrogen was bubbled through the solution for 10 min to purge the excess hydrogen sulfide. The solution was then rotary evaporated carefully (some H₂S outgassing) to remove most of the THF and the residue was partitioned between ethyl acetate and 1 N HCl. The organic phase was washed with water and brine and dried over Na₂SO₄. Evaporation in vacuo to gave 1.07 g of a waxy yellow solid.

¹H-NMR (500 Mz, CD₃OD): δ 1.36 (d, J=7.3 Hz, 3H), 4.25 (q, J =7.3 Hz, 1H), 4.55-4.65 (m, 2H), 5.15-5.35 (m, 2H), 5.9-6.0 (m, 1H).

Step D

A solution of the alloc-D-thioalanine product of Step C (about 1.07 g, 5.65 mmol) in 30 mL of diethyl ether is stirred while dicyclohexylamine (1.13 mL, 5.65 mmol) is added dropwise. After the addition is complete, the thick mixture is stirred for about 15 min more and then allowed to stand for 1 hour. The solid is isolated by filtration, washing with 8 mL of diethyl ether, and drying in vacuo to give about 1.65 g of a white solid. Recrystallization from ethyl acetate gives about 1.21 g of alloc-D-thioalanine dicyclohexylamine salt as colorless needles.

¹H-NMR (500 Mz, CD₃OD): δ 1.15-1.45 (m, 13H), 1.7-1.8 (m, 2H), 1.85-1.95 (m, 4H), 2.05-2.15 (m, 4H), 3.15-3.25 (m, 2H), 4.25 (m, 1H), 4.5-4.6 (m, 2H), 5.15-5.35 (m, 2H), 5.85-5.95 (m, 1H).

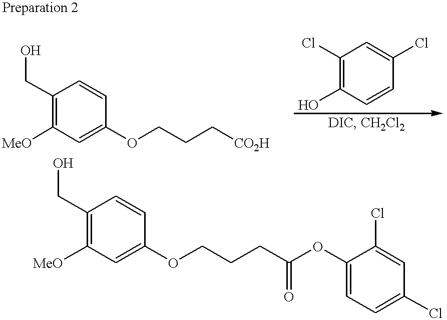

2,4-dichlorophenyl 4-(4-hydroxy-methyl-3-methoxyphenoxy)-butyrate

To a suspension of 4-(4-hydroxy-methyl-3-methoxyphenoxy)-butyric acid (5.01 g, 20.9 mmol) and 2,4-dichlorophenol (4.43 g, 27.2 mmol) in 70 mL of CH₂Cl₂ is added neat 1,3-diisopropylcarbodiimide (3.92 mL, 25.0 mmol). A clear solution is briefly obtained, and then a precipitate will begin to form. After about 3 hours, 70 mL of diethyl ether is added and the mixture is stirred for about 1 hour before filtration. Flash chromatography on silica gel (1:1 EtOAc/hexane) gives about 7.17 g of the inventive compound as a white solid.

¹H-NMR (500 Mz, CDCl₃): δ 2.2-2.3 (m, 2H), 2.87 (t, J=7.3 Hz, 2H), 3.86 (s, 3H), 4.11 (t, J=5.8 Hz, 2H), 4.63 (d, J=4.8 Hz, 2H), 6.47 (dd, J=8.3, 2.1 Hz, 1H), 6.50 (d, J=2.1 Hz, 1H), 7.09 (dd, J=8.7, 0.7 Hz, 1H), 7.18 (d, J=8.2 Hz, 1H), 7.25-7.30 (m, 1H), 7.47 (d, J=0.7 Hz, 1H).

### EXAMPLE 1

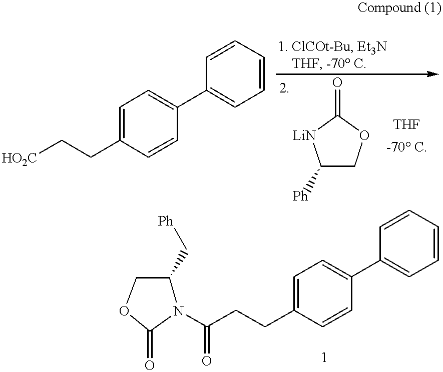

To a stirred solution of 3-(4-biphenyl)-propionic acid (1.997 g, 8.825 mmol) in 40 mL of THF was added to Et₃N (1.4 mL, 10.0 mmol) and the solution was cooled to −70° C. Neat pivaloyl chloride (1.1 ml, 8.9 mmol) was added to this solution and a thick white suspension resulted. After 15 min, the reaction mixture was warmed by placement in an ice bath and kept at 0° C. for 40 min. The mixture was then re-cooled to −70° C. In a separate flask, a solution of (4S)-benzyl-2-oxazolidinone (1.564 g, 8.825 mmol) in 40 mL of THF was cooled to −70° C. and metalated by the dropwise addition of a 2.5M solution of n-butyllithium in hexanes (3.53 mL, 8.825 mmol). The resulting anion solution was added to the re-cooled suspension via a cannula, rinsing with an additional 2.5 mL of THF. After 15 min, the reaction mixture was warmed by placing in an ice bath and kept at 0° C. for 45 min. The reaction mixture was hydrolyzed by the addition of sat. aqueous NH₄Cl and most of the THF was removed by rotary evaporation. The residue was partitioned between ethyl acetate and sat. aqueous NH₄Cl, and the organic phase was washed with sat. aqueous NaHCO₃, water and brine. The organic layer was dried over Na₂SO₄ and evaporated in vacuo to produce a solid. Flash chromatography through 200 g of silica gel (CH₂Cl₂) yielded 2.625 g of Compound 1 as a white solid.

¹H-NMR (500 Mz, CDCl₃): δ 2.79 (dd, J=13.3, 9.4 Hz, 1H), 3.08-3.13 (m, 2H), 3.27-3.41 (m, 3H), 4.16-4.23 (m, 2H), 4.68-4.72 (m, 1H), 7.17-7.61 (M, 14H).

MS (CI): m/z=386.1 (MH+).

### EXAMPLE 2

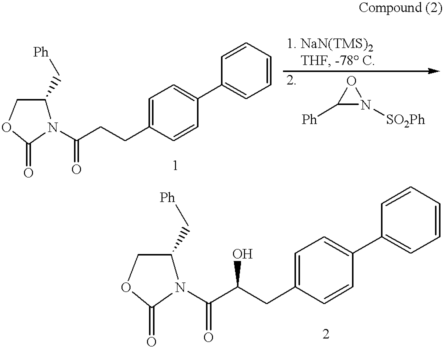

A 1.0 M solution of NaN(TMS)₂ in THF (8.2 mL, 8.2 mmol) was diluted with 45 mL of THF and cooled to −78° C. To this cooled solution was added dropwise a solution of compound 2 (2.625 g, 6.810 mmol) in 100 mL of THF during 20 min. After 25 min, a solution of 2-(phenylsulfonyl)-3-phenyloxaziridine (2.67 g, 10.2 mmol) in 15 mL of THF was added dropwise during 7 min. The solution was stirred at −78° C. for 75 min and was then quenched with a 2.0 M solution of HOAc in THF (10.2 mL, 20.4 mmol). After 5 min, the cooling bath was removed and the reaction mixture was allowed to warm for 20 min. The reaction mixture was then hydrolyzed by the addition of water and extracted with EtOAc. The organic layer was washed with sat. aqueous NaHCO₃, water and brine, and then dried over Na₂SO₄. Evaporation gave a foam which was flash chromatographed though silica gel (2.5% Et₂O/CH₂Cl₂) to give 1.93 g of Compound 2 as a white solid.

¹H-NMR (500 Mz, CDCl₃): δ 2.88 (dd, J=13.4, 9.6 Hz, 1H), 2.98 (dd, J=13.7, 8.0 Hz, 1H), 3.25 (dd, J=13.7, 4.1 Hz, 1H), 3.34 (dd, J=13.5, 3.0 Hz, 1H), 3.56 (d, J=7.7 Hz, 1H), 4.25-4.29 (bs, 2H), 4.64-4.68 (m, 1H), 5.32-5.37 (m, 1H), 7.2-7.7 (m, 14H).

MS (ESI): m/z=419.2 (M+NH₄+), 402.4 (MH+).

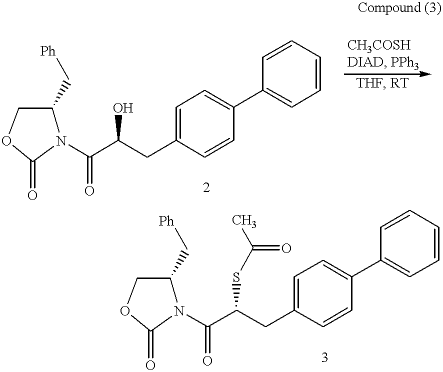

To a solution of PPh₃ (159 mg, 0.61 mmol) in 2 mL of THF at 0° C. was added diisopropyl azodicarboxylate (0.120 mL, 0.61 mmol) dropwise. The resulting pale yellow suspension was stirred at 0° C. for 30 min, and then a solution of [alcohol] Compound 2 (121.5 mg, 0.3026 mmol) and thioacetic acid (0.043 mL, 0.61 mmol) in 1.5 mL of THF was added dropwise. After 1 hour, the cooling bath was removed and the reaction was allowed to proceed for 2.5 hours at room temperature. The reaction mixture was evaporated in vacuo, and the residue was flash chromatographed through silica gel (2.5% Et₂O/CH₂Cl₂) to yield 139 mg of Compound 3 as a foam.

¹H-NMR (500 Mz, CDCl₃): δ 2.32 (s, 3H), 2.62 (dd, J=13.3, 9.4 Hz, 1H), 3.05 (dd, J=13.5, 8.2 Hz, 1H), 3.15 (dd, J=13.5, 3.2 Hz, 1H), 3.43 (dd, J=13.5, 7.3 Hz, 1H), 4.15 (ddd, J=8.9, 1.2, 1.2 Hz, 1H), 4.27 (dd, J=8.7, 8.3 Hz, 1H), 4.65-4.75 (m, 1H), 5.83 (dd, J=8.2, 7.6 Hz, 1H), 7.09 (d, J=7.6 Hz, 2H), 7.2-7.6 (m, 12H).

MS (CI): m/z=477.2 (M+NH₄+), 460.1 (MH+).

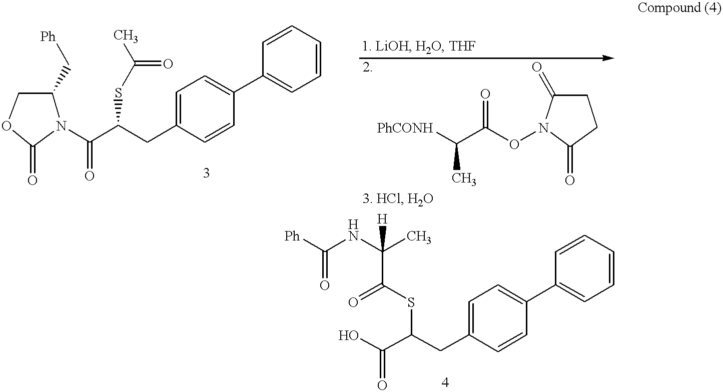

A solution of the starting material, Compound 3, (67.5 mg, 0.147 mmol) in 1.5 mL of THF was cooled via cooling bath to 10° C. and a 0.53 M solution of aqueous LiOH (0.70 mL, 0.37 mmol) was added dropwise. After several minutes, the cooling bath was removed. After 1.5 hour, the solution was adjusted to pH 8 by addition of 1.0 N aqueous HCl. N-benzoyl-D-alanine N-hydroxysuccinimide ester (55 mg, 0.19 mmol) was added as a solid and then the solution was re-adjusted to pH>7 by addition of 0.53 M aqueous LiOH. After 30 min, the solution was acidfied with 1.0 N aqueous HCl and extracted with EtOAc. The organic layer was washed with water and brine, and dried over sodium sulfate. Evaporation in vacuo gave an oil which was purified by reverse phase medium pressure chromatography on RP-18 (1:1 MeCN/0.1% aqueous TFA) to give, after lyophilization, 29 mg of Compound 4 as a ˜1.7:1 mixture of diastereomers.

¹H-NMR (500 Mz, CD₃OD): δ 1.41 (d, J=7.2 Hz, 3H, isomer A, major), 1.45 (d, J=7.2 Hz, 3H, isomer B, minor), 3.00-3.07 (m, 1H, isomers A & B), 3.21-3.31 (m, 1H, isomers A & B), 4.35-4.38 (m, 1H, isomers A & B), 4.72-4.77 (m, 1H, isomers A & B), 7.28-7.57 (m, 12H, isomers A & B), 7.84-7.87 (m, 2H, isomers A & B).

MS (ESI): m/z=451.2 (M+NH₄+), 434.3 (MH+).

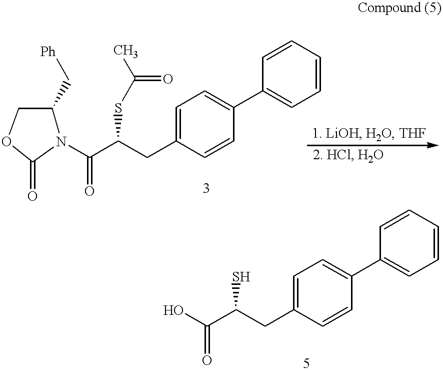

A solution of the starting material, Compound 3, (24.9 mg, 0.0542 mmol) in 0.55 mL of THF was cooled, via cooling bath, to 10° C. and a 0.60 M solution of aqueous LiOH (0.27 mL, 0.16 mmol) was added dropwise. After 1 minute, the cooling bath was removed. After 2.5 hours, the solution was acidfied with 1.0 N aqueous HCl and extracted with EtOAc. The organic layer was washed with water and brine, and dried over sodium sulfate. Evaporation in vacuo gave an oil which was purified by reverse phase medium pressure chromatography on RP-18 (1:1 MeCN/0.1% aqueous TFA) to give after lyophilization 6.2 mg of Compound 5 as a white solid.

¹H-NMR (500 Mz, CDCl₃): δ 2.22 (d, J=8.4 Hz, 1H), 3.09 (dd, J=14.0, 6.9 Hz, 1H), 3.33 (dd, J=14.0, 8.3 Hz, 1H), 3.65-3.75 (m, 1H) 7.28-7.60 (m, 9H).

MS (EI): m/z=258.1 (M+).

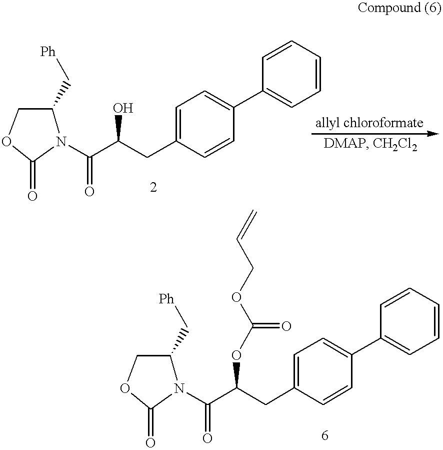

A solution of the starting material 2 (1.81 g, 4.51 mmol) in 40 mL of CH₂Cl₂ was cooled to 0° C. and N,N-dimethylaminopyridine (0.88 g, 7.2 mmol) was added followed by allyl chloroformate (0.720 mL, 6.79 mmol). After 1 hour, the reaction mixture was partitioned between EtOAc and sat. aqueous NH₄Cl. The organic layer was washed with water and brine and dried over sodium sulfate. Evaporation in vacuo gave 2.2 g of Compound 6 as a colorless foam.

¹H-NMR (500 Mz, CDCl₃): δ 2.89 (dd, J=13.5, 9.4 Hz, 1H), 3.12 (dd, J=13.7, 9.3 Hz, 1H), 3.25-3.35 (m, 2H), 4.14-4.25 (m, 2H), 4.60-4.70 (m, 3H), 5.25-5.40 (m, 2H), 5.9-6.9 (m,1H), 6.21 (dd, J =9.4, 3.4 Hz, 1H), 7.25-7.61 (m, 14H).

MS (CI): m/z=503.2 (M+NH₄+).

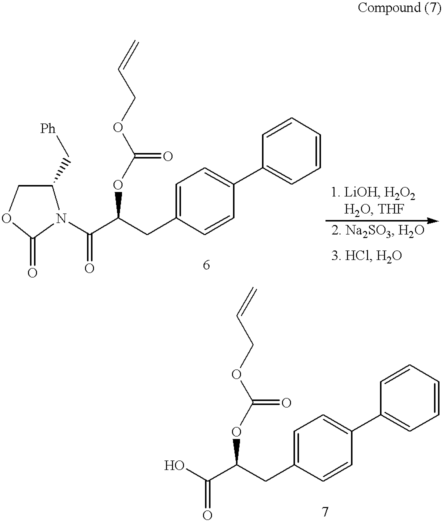

A solution of the starting material, Compound 6, (2.2 g, 4.51 mmol) in 35 mL of 4:1 THF/H₂O was cooled to 0° C. and 30% hydrogen peroxide (1.84 mL, 18 mmol) was added followed by dropwise addition of 1.0 M aqueous LiOH (7.2 mL, 7.2 mmol). After 35 minutes, a 1.5 M solution of aqueous Na₂SO₃ (12 mL, 18 mmol) was added. The solution was acidfied with 1.0 N aqueous HCl and extracted with EtOAc. The organic layer was washed with water and brine, and dried over sodium sulfate. Evaporation in vacuo gave the crude product which was purified by flash chromatography on silica gel (CH₂Cl₂/MeOH/HOAc) to give 0.739 g of product, Compound 7, as a white solid.

¹H-NMR (500 Mz, CDCl₃): δ 3.22 (dd, J=14.6, 8.9 Hz, 1H), 3.33 (dd, J=14.6, 3.9 Hz, 1H), 4.6-4.7 (m, 2H), 5.24-5.38 (m, 3H), 5.89-5.95 (m, 1H), 7.35-7.65 (m, 9H).

MS (CI): m/z=344.1 (M+NH₄+).

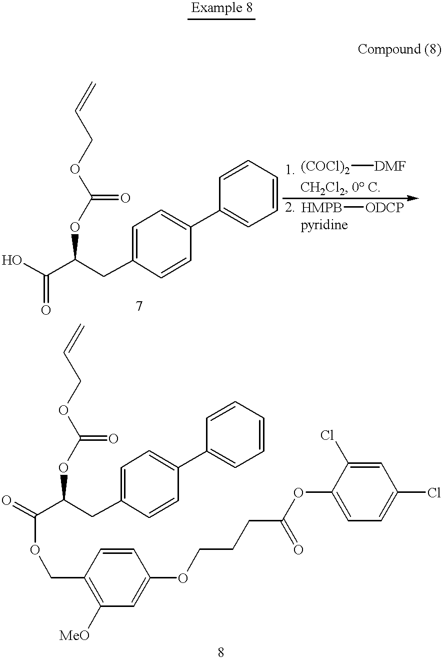

A 2.0 M solution of oxalyl chloride in CH₂Cl₂ (0.720 mL, 1.44 mmol) was added dropwise to a solution of DMF in CH₂Cl₂ (0.152 mL, 1.96 mmol) which had been cooled to 0° C. The resulting white suspension was vigorously stirred while a solution of starting material, Compound 7, (0.4275 g, 1.310 mmol) in 4 mL of CH₂Cl₂ was added dropwise giving a colorless solution. After 5 minutes, pyridine (0.106 mL, 1.31 mmol) was added followed by a solution of 2,4-dichlorophenyl 4-(4-hydroxy-methyl-3-methoxyphenoxy)-butyrate (0.556 g, 1.44 mmol) and pyridine (0.159 mL, 1.97 mmol) in 4 mL of CH₂Cl₂. After 5 minutes, the reaction mixture was partitioned between EtOAc and sat. aqueous NH₄Cl. The organic layer was washed with saturated aqueous NaHCO₃, water and brine, and dried over sodium sulfate. Evaporation in vacuo gave the crude product which was purified by flash chromatography on silica gel (100:1:0.1 CH₂Cl₂/EtOAc/Et₃N) to give 0.705 g of product, Compound 8, as an oil.

¹H-NMR (500 Mz, CDCl₃): δ 2.2-2.3 (m, 2H), 2.86 (t, J=7.3Hz, 2H), 3.18 (dd, J=14.4, 8.2 Hz, 1H), 3.25 (dd, J=14.4, 4.3 Hz, 1H), 3.82 (s, 3H), 4.07 (t, J=6.1 Hz, 2H), 4.60-4.65 (m, 2H), 5.16 (d, J=11.7 Hz, 1H), 5.25 (d, J=11.7 Hz, 1H), 5.2-5.4 (m, 3H), 5.85-5.95 (m, 1H), 6.43 (dd, J=8.2, 2.3 Hz, 1H), 6.44 (d, J=2.3 Hz, 1H), 7.10 (d, J=8.7 Hz, 1H), 7.16 (d, J=8.2 Hz, 1H), 7.25-7.60 (m, 11H).

MS (CI): m/z=710.4 (M+NH₄+).

[chemical structure]

Rapp TentaGel S—NH₂ resin (0.25 mmol/g, 1.150 g, 0.288 mmol) was swelled with dry DMF in a 12 mL solid phase extraction cartridge. The resin was washed with dry DMF (4×4 mL) and then drained. Starting material, Compound 8, (0.450 g, 0.649 mmol), 1-hydroxy-benzotriazole (0.088 g, 0.65 mmol) and diisopropylethylamine (0.113 mL, 0.65 mmol) were dissolved in DMF (4 mL) and the solution was added to the drained resin. The resin-solution was mixed for 17 hours, at which point a Kaiser test on a small sample of the resin-solution yielded negative results. The resin-solution was drained and washed with DMF (3×4 mL). These washes were saved for later recovery of the excess starting material, Compound 8. To the drained-resin was added a solution of acetic anhydride (0.136 mL, 1.44 mmol) and pyridine (0.140 mL, 1.73 mmol) in 4 mL of DMF, and the drained-resin was mixed for 1 hour and again drained. This final resin was then washed as follows: DMF (4×5 mL), THF (4×5 mL), MeOH (4×5 mL), CH₂Cl₂ (5×5 mL). The final resin was dried briefly under a stream of nitrogen and then in vacuo giving a final weight of 1.284 g of Resin 9. Cleavage of substrate from a weighed portion of Resin 9 with 5% TFA/ CH₂Cl₂ allowed the new titer of the resin to be determined as 0.20 mmol/g.

The saved DMF washes from above were diluted with EtOAc and washed with sat. aqueous NH₄Cl, water and brine, and dried over sodium sulfate. Evaporation in vacuo gave 0.289 g of recovered starting material, Compound 8, which contained some 2,4-dichlorophenol.

[chemical structure]

Resin 9 (0.20 mmol/g, 1.182 g, 0.2365 mmol) was swelled with dry N-methylpyrrolidinone (NMP) and then washed with NMP (3×5 mL) and drained. To a solution of Pd(PPh₃)₄ (0.055 g, 0.048 mmol) in 4 mL of NMP was added acetic acid (0.140 mL, 2.45 mmol) followed by N-methylmorpholine (0.265 mL, 2.41 mmol) and this solution was added to the above drained Resin 9. Resin 9 was mixed, and significant outgassing was noted during the first 5 minutes. After 3 hours, the resin was drained and then washed as follows: NMP (4×5 mL), 3% Et₂NCS₂Na/NMP (1×5 mL), NMP (1×5 mL), DMF (4×5 mL), THF (4×5 mL), MeOH (4×5 mL), CH₂Cl₂ (6×5 mL). Resin 9 was dried briefly under a stream of nitrogen and then in vacuo giving a final weight of 1.164 g of Resin 10.

[chemical structure]

Resin 10 (0.20 mmol/g, 0.551 g, 0.110 mmol) was swelled with 5 mL of dry THF under nitrogen in a solid phase reaction cartridge and then washed 4×3 mL with dry THF. In a separate flask, tris(4-chlorophenyl)phosphine (0.202g, 0.552 mmol) was dissolved in 2 mL of THF, cooled, via cooling bath, to 0° C. and diisopropyl azodicarboxylate (0.109 mL, 0.552 mmol) was added dropwise during 5 minutes. The cooling bath was removed and the yellow solution was stirred for 15 minutes. Recrystallized alloc-D-thioalanine dicyclohexylamine salt (0.205 g, 0.552 mmol) was added thereto which it dissolved with stirring during 2 to 3 minutes. The resulting light yellow solution was added to the above drained Resin 10 and the reaction was mixed for 2.75 hours at room temperature. The solution was drained and the Resin 10 was washed with THF (4×), DMF (4×), THF (4×), MeOH (4×) and CH₂Cl₂ (6×). Resin 10 was dried briefly under a stream of nitrogen and then in vacuo giving a final weight of 0.576 g of Resin 11.

[chemical structure]

Resin 11 (0.20 mmol/g, 0.024 g, 0.0048 mmol) was swelled with 0.5 mL of dry CH₂Cl₂ under nitrogen and then washed 3×0.5 mL with dry CH₂Cl₂. To the drained Resin 10 was added 0.1 mL of a 0.5M solution of acetic anhydride in CH₂Cl₂ (10 eq). This was followed after 1 minute by addition of 0.1 mL of a CH₂Cl₂ solution containing 0.25 eq of Pd(PPh₃)_{4,} 0.5 eq of PPh₃ and 5 eq of PhSiH₃. The reaction was allowed to proceed at room temperature for 1 hour, mixing periodically, and some gas evolution was observed. The resin was drained and washed with CH₂Cl₂ (3×), DMF (3×), THF (3×), MeOH (3×), and CH₂Cl₂ (4×). The resin was dried briefly under a stream of nitrogen and then in vacuo giving Resin 12.

[chemical structure]

Resin 12 (0.024 g, 0.0048 mmol) was swelled with 0.5 mL of dry CH₂Cl₂ under nitrogen and then washed 3×0.5 mL with dry CH₂Cl₂. The product was cleaved from the resin with 5% TFA/CH₂CI₂ (5×0.25 mL, 2 min each) and the combined solutions were evaporated to give 2.3 mg of an oil. Lyophilization from 1:1 MeCN/water gave 1.9 mg of thioester, Compound 13, as a pale yellow solid.

¹H-NMR (500 Mz, CD₃OD): δ 1.27 (d, J=7.1 Hz, 3H), 1.98 (s, 3H), 3.01 (dd, J=14.0, 7.4 Hz, 1H), 3.28 (dd, J=14.0, 8.0 Hz, 1H), 4.34 (t, J=7.5 Hz, 1H), 4.48 (q, J=7.1 Hz, 1H), 7.25-7.35 (m, 3H), 7.41 (dd, J=7.8, 7.5 Hz, 2H), 7.52 (d, J=8.1 Hz, 2H), 7.57 (d, J=7.3 Hz, 2H).

MS (ESI): m/z=389.3 (M+NH₄+).

[chemical structure]

Resin 10 (0.20 mmol/g, 0.075 g, 0.015 mmol) was swelled with 1 mL of dry THF under nitrogen in a solid phase reaction cartridge and then washed 4×1 mL with dry THF and drained. In a separate flask tris(4-chlorophenyl)phosphine (0.219g, 0.60 mmol) was dissolved in 3 mL of THF, cooled to 0° C., via cooling bath, and diisopropyl azodicarboxylate (0.118 mL, 0.60 mmol) was added dropwise during 5 minutes. The cooling bath was removed and the yellow solution was stirred for 15 minutes. Thioacetic acid (0.043 mL, 0.60 mmol) was added and the solution was stirred for 2 to 3 minutes. A 0.60 mL portion of the resulting light yellow solution (˜8 equiv.) was added to the above drained resin followed by N,N-diisopropylethylamine (0.026 mL, 0.15 mmol) and the solution was mixed for 3 hours at room temperature. The solution was drained and the resin was washed with THF (4×), DMF (4×), THF (4×), MeOH (4×) and CH₂Cl₂ (6×). The resin was dried briefly under a stream of nitrogen and then in vacuo giving Resin 14.

[chemical structure]

Resin 14 (0.075 g, 0.015 mmol) was swelled with 1.0 mL of dry CH₂Cl₂ under nitrogen and then washed 3×0.5 mL with dry CH₂Cl₂. The product was cleaved from the resin with 5% TFA/CH₂Cl₂ (5×0.5 mL, 2 min each) and the combined solutions were evaporated to give the Compound 15 as an oil.

¹H-NMR (500 Mz, CD₃OD): δ 2.03 (s, 3H), 3.02 (dd, J=14.0, 7.1 Hz, 1H), 3.25 (dd, J=14.0, 8.3 Hz, 1H), 4.39 (t, J=7.5 Hz, 1H), 7.25-7.60 (m, 9H).

MS (CI): m/z=318.2 (M+NH₄+).

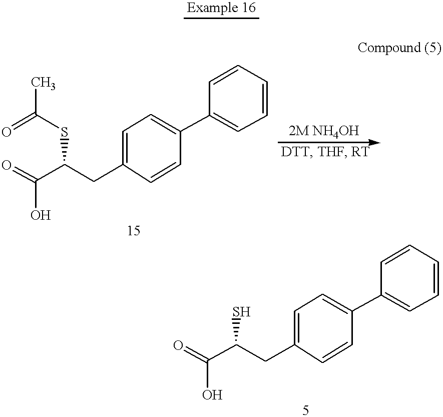

To a solution of the starting material Compound 15, (3.4 mg, 0.011 mmol) and dithiothreitol (2.0 mg, 0.013 mmol) in 0.3 mL of THF was added 2 M aqueous NH₄OH (0.3 mL, 0.6 mmol). After 1 hour, the solution was acidfied with 1.0 N aqueous HCl and extracted with EtOAc. The organic layer was washed with water and brine, and dried over sodium sulfate. Evaporation in vacuo gave a white solid which was purified by reverse phase medium pressure chromatography on RP-18 (55:45 MeCN/0.1% aqueous TFA) to give, after lyophilization, 2.8 mg of Compound 5 as a white solid. The spectral properties of this compound agreed with those obtained for Compound 5 prepared according to Example 5.

[chemical structure]

Resin 10 (0.20 mmol/g, 0.096 g, 0.019 mmol) was swelled with 1 mL of dry THF under nitrogen in a solid phase reaction cartridge and then washed 4×1 mL with dry THF and drained. A THF solution (0.8 mL) containing 8 equivalents of formic acid and 8 equivalents of PPh3 was added to the resin followed by dropwise additon of diisopropyl azodicarboxylate (0.031 mL, 0.16 mmol, 8 equiv.) to provide a reaction mixture, which was mixed for 3.5 hours at room temperature. The solution was drained and the resin was washed with THF (4×), DMF (4×), THF (4×), MeOH (4×) and CH₂Cl₂ (6×). The resin was dried briefly under a stream of nitrogen and then in vacuo.

Resin 10 was re-swelled with 1 mL of dry THF under nitrogen and then washed 4×1 mL with dry THF and drained. A 1:1 THF-DMF solution (0.8 mL) containing 8 equivallents of N,N-diisopropylethylamine and 8 equiv. of hydroxylamine hydrochloride was added thereto and the preparation was mixed for 20 hours at room temperature. The solution was drained and the resin was washed with DMF (4×), THF (4×), MeOH (4×) and CH₂Cl₂ (6×). The resin was dried briefly under a stream of nitrogen and then in vacuo to give Resin 17.

[chemical structure]

Resin 17 (0,20 mmol/g, 0.024 g, 0.0048 mmol) was swelled with 0.5 mL of dry THF under nitrogen in a solid phase reaction cartridge and then washed 4×0.5 mL with dry THF and drained. In a separate flask tris(4-chlorophenyl)phosphine (0.0.037 g, 0.10 mmol) was dissolved in 0.5 mL of THF, cooled to 0° C., via cooling bath, and diisopropyl azodicarboxylate (0.0 20 mL, 0.10 mmol) was added dropwise. The cooling bath was removed and the yellow solution was stirred for 15 minutes. Thiobenzoic acid (0.012 mL, 0.10 mmol) was added and the solution was stirred for 2-3 min. A 0.30 mL portion of the resulting light yellow solution (˜12 equiv.) was added to the above drained resin followed by N,N-diisopropylethylamine (0.012 mL, ˜15 equiv.) and the reactants was mixed for 4.5 hours at room temperature. The solution was drained and the resin was washed with THF (4×), DMF (4×), THF (4×), MeOH (4×) and CH₂Cl₂ (6×). The resin was dried briefly under a stream of nitrogen and then in vacuo giving Resin 18.

[chemical structure]

Resin 18 (0.024 g, 0.0048 mmol) was swelled with 0.5 mL of dry CH₂Cl₂ under nitrogen and then washed 3×0.5 mL with dry CH₂Cl₂. The product was cleaved from the resin with 5% TFA/CH₂Cl₂ (5×0.5 mL, 2 minutes each) and the combined solutions were evaporated to give an oil. Purification by reverse phase medium pressure chromatography on RP-18 (60:40 MeCN/0.1% aqueous TFA) gave after lyophilization 1.5 mg of Compound 19 as a white solid.

¹H-NMR (500 Mz, CD₃OD): δ 3.17 (dd, J=14.0, 6.9 Hz, 1H), 3.36 (dd, J=14.0, 8.3 Hz, 1H), 4.62 (t, J=7.6 Hz, 1H), 7.25-7.65 (m, 12H), 7.92 (d, J=7.3 Hz, 1H).

MS (CI): m/z=363.3 (MH+).

### EXAMPLE 20

[chemical structure]

Resin 20B (0.20 mmol/g) was prepared starting from the propionic acid derivative 20A following the procedures described in Examples 1, 2, 6-10 and 14. A portion of Resin 20B (0.023 g, 0.0048 mmol) was swelled with 0.5 mL of dry THF under nitrogen in a solid phase reaction cartridge and then washed 4×0.5 mL with dry THF and drained. A 1:1 THF-DMF solution (0.35 mL) containing 14 equivalents of N,N-diisopropylethylamine and 14 equivalents of hydroxylamine hydrochloride was added and the reactants was mixed for 2 hours at room temperature. The solution was drained and the resin was washed with dry DMF (3×), dry THF (3×) and dry DMF (4×). In a separate flask 4-biphenylacetic acid (0.023 g, 0.11 mmol), and 1-hydroxy-7-azabenzotriazole (0.015 g, 0.11 mmol) were dissolved in 1 mL of DMF and 1,3-diisopropylcarbodiimide (0.017 mL, 0.11 mmol) was added dropwise. After 5 minutes, N,N-diisopropylethylamine (0.019 mL. 0.11 mmol) was added to the solution. A 0.40 mL portion of the solution (˜9 equivalents) was added to the above drained resin and the reactants were mixed for 16 hours at room temperature. The solution was drained and the resin was washed with DMF (4×), THF (4×), MeOH (4×) and CH₂Cl₂ (6×). The product was cleaved from the resin with 5% TFA/CH₂Cl₂ (5×0.5 mL, 2 minutes each) and the combined solutions were evaporated to give an oil. Purification by reverse phase medium pressure chromatography on RP-18 (75:25 MeCN/0.1% aqueous TFA) gave after lyophilization 1.4 mg of Compound 20 as a white solid.

¹H-NMR (500 Mz, CD₃OD): δ 3.12 (dd, J=14.2, 8.3 Hz, 1H), 3.42 (dd, J=14.2, 7.3 Hz, 1H), 3.83 (AB_{q}, J_{AB}=15.0 Hz, Δυ_{AB}=21.6 Hz, 2H), 4.49 (t, J=7.7 Hz, 1H), 7.15 (d, J=8.0 Hz, 2H), 7.25-7.55 (m, 12H), 7.82, (s, 1H), 7.95 (d, J=7.7 Hz, 1H).

### EXAMPLES 21-90

Employing the procedures described herein above, additional compounds of the present invention were prepared. These compounds, defined as R¹, R² and R⁵ moieties, defined herein above, are described in Tables 1 through 7, which also includes characterizing data.

TABLE 1

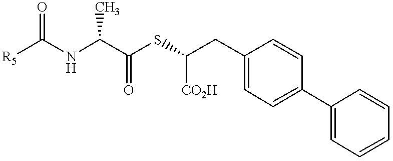

| Example No. | R⁵ | m/z |
|---|---|---|
| 21 | CH₃CH₂— | 403.3 (M + NH₄⁺); ESI |
| 22 | CH₃CH₂CH₂— | 417.3 (M + NH₄⁺); ESI |
| 23 | CH₃(CH₂)₃— | 431.5 (M + NH₄⁺); ESI |
| 24 | HO₂C(CH₂)₂— | 447.3 (M + NH₄⁺); ESI |
| 25 | H₂C═CHCH₂O— | 431.3 (M + NH₄⁺); ESI |
| 26 | (CH₃)₂CHCH₂— | 431.3 (M + NH₄⁺); ESI |
| 27 | (CH₃)₂CH— | 417.2 (M + NH₄⁺); ESI |
| 28 | CH₃(CH₂)₄— | 445.4 (M + NH₄⁺); ESI |
| 29 | HO₂CCH₂SCH₂— | 478.9 (M + NH₄⁺); ESI |
| 30 | (E)-CH₃CH═CH— | 415.0 (M + NH₄⁺); ESI |
| 31 | HO₂C(CH₂)₃— | 461.2 (M + NH₄⁺); ESI |
| 32 | Ph— | 451.3 (M + NH₄⁺); ESI |
| 33 | PhOCH₂— | 481.2 (M + NH₄⁺); ESI |
| 34 | PhCH₂— | 465.3 (M + NH₄⁺); ESI |
| 35 | PhCH₂CH₂— | 479.3 (M + NH₄⁺); ESI |
| 36 | (E)-PhCH═CH— | 477.3 (M + NH₄⁺); ESI |
| 37 | PhCOCH₂CH₂— | 507.1 (M + NH₄⁺); ESI |
| 38 | PhCONHCH₂— | 508.0 (M + NH₄⁺); ESI |
| 39 | 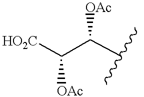 | 563.1 (M + NH₄⁺); ESI |
| 40 | 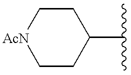 | 483.0 (M + NH₄⁺); ESI |
| 41 | 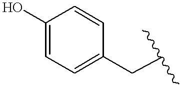 | 481.0 (M + NH₄⁺); ESI |
| 42 | 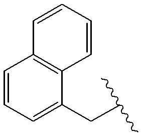 | 515.4 (M + NH₄⁺); ESI |
| 43 | 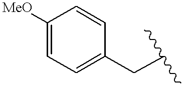 | 495.1 (M + NH₄⁺); ESI |
| 44 | 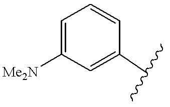 | 477.0 (MH⁺); ESI |
| 45 | 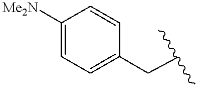 | 491.1 (MH⁺); ESI |
| 46 | 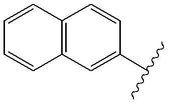 | 501.1 (M + NH₄⁺); ESI |
| 47 | 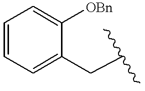 | 571.1 (M + NH₄⁺); ESI |
| 48 | 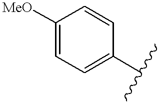 | 481.2 (M + NH₄⁺); ESI |
| 49 | 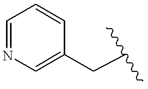 | 449.0 (MH⁺); ESI |
| 50 | 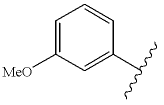 | 481.1 (M + NH₄⁺); ESI |
| 51 | 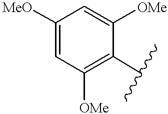 | 441.4 (M + NH₄⁺); ESI |

TABLE 2

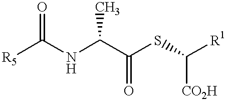

| Example No. | R⁵ | R¹ | m/z |
|---|---|---|---|
| 52 | Me— | 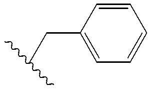 | 313.2 (M + NH₄⁺); ESI |
| 53 | H₂C═CHCH₂O— | 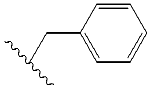 | 355.2 (M + NH₄⁺); ESI |
| 54 | Ph— | 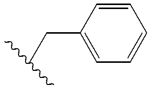 | 375.2 (M + NH₄⁺); ESI |
| 55 | Ph— | Ph— | 361.1 (M + NH₄⁺); ESI |
| 56 | Ph— | 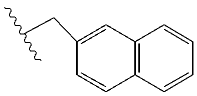 | 425.1 (M + NH₄⁺); ESI |
| 57 | Me— | 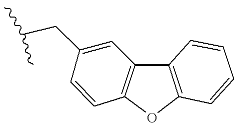 | 403.1 (M + NH₄⁺); ESI |
| 58 | Ph— | 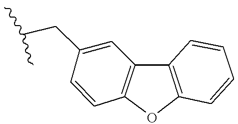 | 470.1 (M + NH₄⁺); ESI |
| 59 | H₂C═CHCH₂O— | 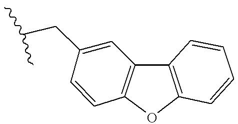 | 445.2 (M + NH₄⁺); ESI |
| 60 | Ph— | 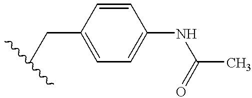 | 432.2 (M + NH₄⁺); ESI |
| 61 | Ph— | 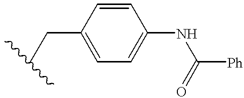 | 494.4 (M + NH₄⁺); ESI |
| 62 | Ph— | 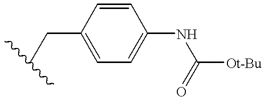 | 490.3 (M + NH₄⁺); ESI |
| 63 | 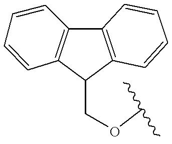 | 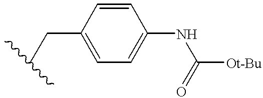 | 608.4 (M + NH₄⁺); ESI |
| 64 | 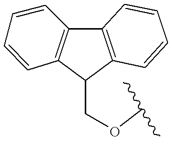 | 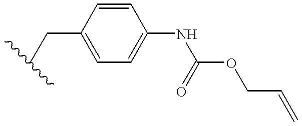 | 592.3 (M + NH₄⁺); ESI |
| 65 | 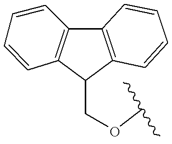 | 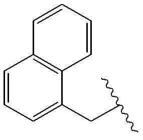 | 493.3 (M + NH₄⁺); ESI |

TABLE 3

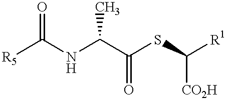

| Example No. | R⁵ | R¹ | m/z |
|---|---|---|---|
| 66 | H₂C═CHCH₂O— | 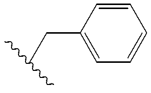 | 355.1 (M + NH₄⁺); ESI |
| 67 | Ph— | 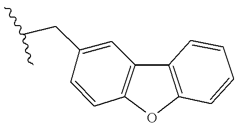 | 465.0 (M + NH₄⁺); ESI |

TABLE 4

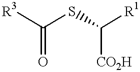

| Example No. | R³ | R¹ | m/z |
|---|---|---|---|
| 68 | CH₃— | 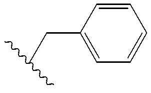 | 242.1 (M + NH₄⁺); ESI |
| 69 | Ph— | 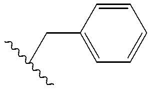 | 304.1 (M + NH₄⁺); ESI |
| 70 | 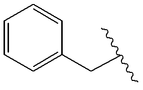 | 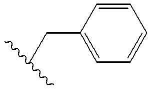 | 318.1 (M + NH₄⁺); ESI |
| 71 | 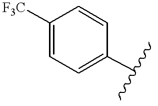 | 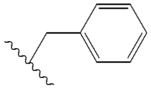 | 354.0 (M⁺); El |
| 72 | 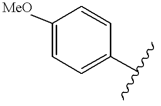 | 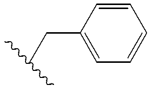 | 334.1 (M + NH₄⁺); ESI |
| 73 | 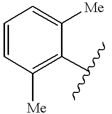 | 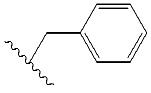 | 314.0 (M⁺); El |
| 74 | 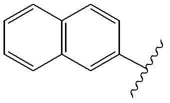 | 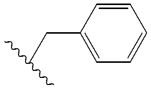 | 336.0 (M⁺); El |
| 75 | 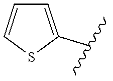 | 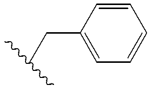 | 292.0 (M⁺); El |
| 76 | Ph— | 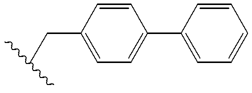 | 380.2 (M + NH₄⁺); Cl |
| 77 | 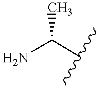 | 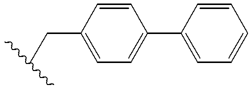 | 330.2 (MH⁺); ESI |
| 78 | CH₃— | 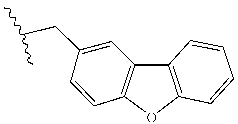 | 314.0 (M⁺); El |
| 79 | Ph— | 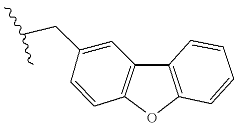 | 376.0 (M⁺); El |
| 80 | Ph— | 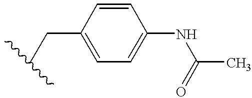 | 361.2 (M + NH₄⁺); Cl |

TABLE 5

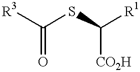

| Example No. | R³ | R¹ | m/z |
|---|---|---|---|
| 81 | CH₃— | 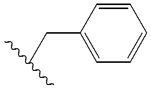 | 242.1 (M + NH₄⁺); Cl |
| 82 | Ph— | 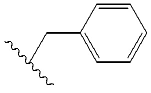 | 304.1 (M + NH₄⁺); Cl |
| 83 | CH₃— | 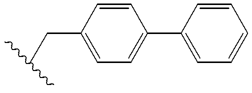 | 318.1 (M + NH₄⁺); Cl |
| 84 | CH₃— | 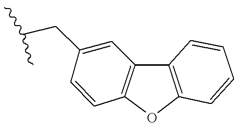 | 332.1 (M + NH₄⁺); Cl |
| 85 | Ph— | 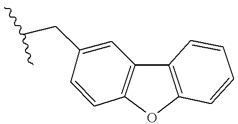 | 394.2 (M + NH₄⁺); Cl |

TABLE 6

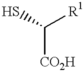

| Example No. | R¹ | m/z |
|---|---|---|
| 86 | 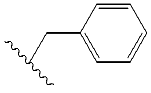 | 182.0 (M⁺); El |
| 87 | 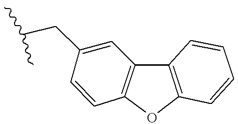 | 272.1 (M⁺); El |

TABLE 7

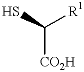

| Example No. | R¹ | m/z |
|---|---|---|
| 88 | 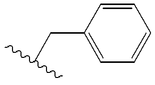 | 149.0 (M − S)H EI |
| 89 | 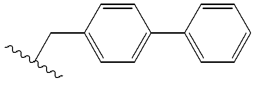 | 276.2 (M + NH₄⁺); Cl |
| 90 | 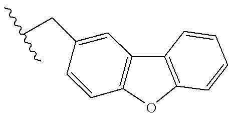 | 290.1 (M + NH₄⁺); Cl |

Biological Activity

IMP-1 metallo-B-lactamase lacking the N-terminal 18 hydrophobic amino acids which encode the putative periplasmic signal sequence (EMBL access code PACATAAC6) was PCR amplified from plasmid DNA prepared from a carbapenem-resistant strain of Pseudomonas aeruginosa (CL5673). The PCR product was cloned into pET30a+ (Novegen) and expressed in E.coli BL21(DE3) after induction with 0.5 mM IPTG for 20 hours at room temperature in minimal media supplemented with casamino acids and 348 μM ZnSO₄. Soluble IMP-1 was purified from cell extracts by SP-Sepharose (Pharmacia) ion exchange and Superdex 75 (Pharmacia) size-exclusion chromatography.

The IC₅₀ of thiol derivatives was determined following a 15 minute incubation at 37° C. with IMP-1 (0.75nM in 50 mM MOPS, pH 7). Using initial velocity as a measure of activity, inhibition was monitored spectrophotometrically at 490 nm in a Molecular Devices SPECTRAmax™ 250 96-well plate reader employing nitrocefin as the reporter substrate at approximately Kₘ concentration (60 μM).

A laboratory strain of E.coli engineered to express IMP-1 was used to evaluate the ability of thiol derivatives to reverse metallo-β-lactamase-mediated carbapenem resistance in bacteria. Native IMP-1, which included the N-terminal periplasmic signal sequence, was PCR amplified from CNA isolated from a carbapenem resistant P. aeruginosa clinical isolate, CL56673, and cloned into the pET30a vector. The basal (uninduced) level of IMP-1 expressed when pET30a-IMP-1 was introduced into E. coli BL21(DE3) resulted in 4-, 64- or 500-fold reduced sensitivity to impenem, meropenem or (1S,5R,6S)-1-methyl-2-{7-[4-(aminocarbonylmethyl)-1,4-diazoniabicyclo(2.2.2)octan-1-yl]methyl-fluoren-9-on-3-yl}-6-(1R-hydroxyethyl)-carbapen-2-em-3-carboxylate chloride (a carbapenem synthesized at Merck Research Laboratories) respectively. For example, the minimum inhibitory concentration (MIC) of (1S,5R,6S)-1-methyl-2-{7-[4-(aminocarbonylmethyl)-1,4-diazoniabicyclo(2.2.2)octan- 1-yl]methyl-fluoren-9-on-3-yl}-6-(1R-hydroxyethyl)-carbapen-2-em-3-carboxylate chloride, was typically increased from 0.06-0.12 μg/ml to 16-32 μg/ml by the expression of IMP-1. To evaluate IMP-1 inhibitors, an overnight culture of E. coli BL2(DE3)/pET30a-IMP-1, grown 35° C. in LB broth (Difco) or Mueller Hinton broth (BBL) supplemented with kanamycin (50 μM/ml), was diluted to a final concentration of ˜10⁵ cells/ml in Mueller Hinton broth (BBL) containing a subinhibitory concentration (0.25× MIC) of the carbapenem, (1S,5R,6S)-1-methyl-2-{7-[4-(aminocarbonylmethyl)-1,4-diazoniabicyclo(2.2.2)octan-1-yl]methyl-fluoren-9-on-3-yl}-6-(1R-hydroxyethyl)-carbapen-2-em-3-carboxylate chloride. Various concentrations of IMP-1 inhibitor were added to the bacterial growth medium and their capacity to effect a four-fold or greater increase in sensitivity to the carbapenem was monitored. The readout for antibacterial activity showed no visible growth after 20 hours incubation at 35° C.

The activity of thiol derivatives, against purified IMP-1 metallo-β-lactamase was tested and found to be active in an IC₅₀ range from about 0.0004 to about 750 μM. Synergy between thiol derivatives and the carbapenem, (1S,5R,6S)-1-methyl-2-{7-[4-(aminocarbonylmethyl)-1,4-diazoniabicyclo(2.2.2)octan-1-yl]methyl-fluoren-9-on-3-yl}-6-(1R-hydroxyethyl)-carbapen-2-em-3-carboxylate chloride, against an IMP-1 producing E. coli bacterial strain is illustrated in Table 8.

TABLE 8

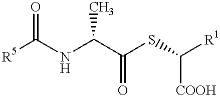

| | | | Effective conc for 4-fold |
|---|---|---|---|
| | | | reduction of MIC in E. coli ^{a} |
| Example No. | R⁵ | R¹ | (μM) |
| 54 | 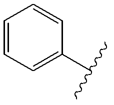 | 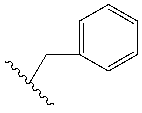 | 25 |
| 32 | 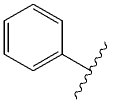 | 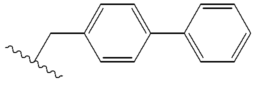 | 6.3 |
| 58 | 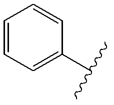 | 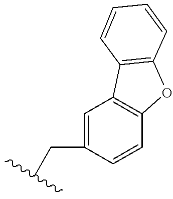 | 3.1 |
| 13 | CH₃ | 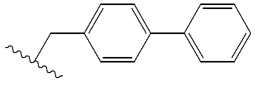 | 6.3 |
| 57 | CH₃ | 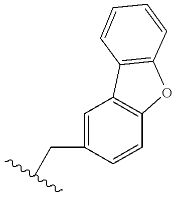 | 3.1 |
| 69 | 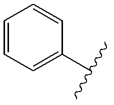 | 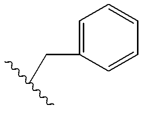 | 50 |
| 76 | 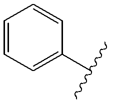 | 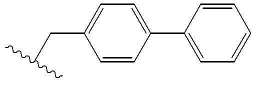 | 3.1 |
| 79 | 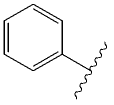 | 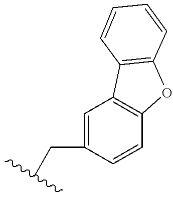 | 0.2 |
| 68 | CH₃ | 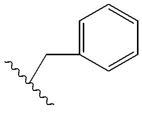 | 12.5 |
| 15 | CH₃ | 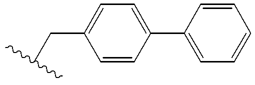 | 25 |
| 78 | CH₃ | 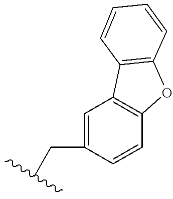 | ≦0.1 |

^{a}Concentration of thioester that produces a 4-fold increase in sensitivity to the carbapenem (1S,5R,6S)-1-methyl-2-{7-[4-(aminocarbonylmethyl)-1,4-diazoniabicyclo(2.2.2)octan-1-yl]methyl-fluoren-9-on-3-yl}-6-(1R-hydroxyethyl)-carbapen-2-em-3-carboxylate chloride in an IMP-1-producing laboratory strain E. coil BL21 (DE3)/pET 30a-IMP-1.

We claim:

1. Thiol derivative compounds, pharmaceutically acceptable salts and biolabile esters thereof, useful for treating bacterial infections of formula I: 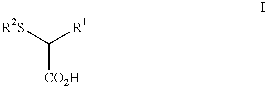
wherein:
R¹ is selected from the group consisting of straight, branched, unsaturated or alicyclic alkyl, optionally substituted with from 1 to 3 R_{X} groups; and (CH₂)ₙAr, where Ar is an aryl selected from the group consisting of phenyl, furanyl, thienyl, pyridyl, naphthyl, biphenyl, dibenzofuranyl, dibenzothienyl, fluorenyl and fluorenonyl, where n is 0, 1, 2 or 3, and where Ar is optionally substituted with 1 to 3 R_{X} groups;
R² is selected from the group consisting of hydrogen; and a group of formula II: 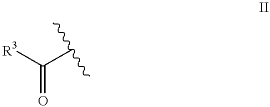
wherein:
R³ is selected from the group consisting of hydrogen; straight, branched, unsaturated or alicyclic alkyl, optionally substituted with from 1 to 3 R_{X} groups; (CH₂)ₙAr, where Ar is an aryl selected from the group consisting of phenyl, furanyl, thienyl, pyridyl, naphthyl, biphenyl dibenzofuranyl, dibenzothienyl, fluorenyl and fluorenonyl, where Ar is optionally substituted with 1 to 3 R_{X} groups, and where n is 0, 1, 2 or 3; and a group of formula III: 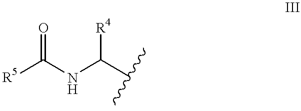
wherein:
R⁴ is selected from the group consisting of hydrogen; and straight or branched alkyl;
R⁵ is selected from the group consisting of hydrogen; straight, branched, unsaturated or alicyclic alkyl, optionally substituted with 1 to 3 R_{X} groups, where the alkyl group is optionally interrupted by X, where X is selected from O, S, NH and N(COCH₃); allyloxy and 9-fluorenylmethyloxy; and (CH₂)ₙAr, where Ar is selected from the group consisting of phenyl, furanyl, thienyl, pyridyl, naphthyl, biphenyl, dibenzofuranyl, dibenzothienyl, fluorenyl and fluorenonyl, where n is 0, 1, 2 or 3, and where Ar is optionally substituted with 1 to 3 R_{X} groups; and
R_{X} is selected from the group consisting of OR, CN, C(O)NH₂, C(O)NHR, C(O)N(R)₂, OC(O)NH₂, OC(O)R, CHO, SO₂NH₂, SOR, CF₃, C(O)R, COOR, F, Cl, Br, I, OCH₂Ph, NHR, N(R)₂, NHCOR, NHCO₂t-Bu, NHCO₂allyl, NH₂, and R, where R is selected from hydrogen, C₁ to C₁₅ alkyl, and aryl.

2. The compound according to claim 1, wherein the derivative is selected from the group consisting of formulae Ia and Ia′:

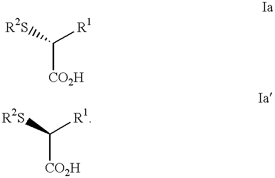

3. The compound according to claim 2, wherein the derivative is of the formula Ia: 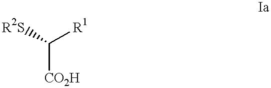
wherein R² is hydrogen.

4. The compound according to claim 2, wherein the derivative is of the formula: 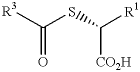
wherein:
R³ is selected from the group consisting of hydrogen; straight, branched, unsaturated or alicyclic alkyl, optionally substituted with from 1 to 3 R_{X} groups; and (CH₂)ₙAr, where Ar is an aryl selected from the group consisting of phenyl, furanyl, thienyl, pyridyl, naphthyl, biphenyl, dibenzofuranyl, dibenzothienyl, fluorenyl and fluorenonyl, where n is 0, 1, 2 or 3, and where Ar is optionally substituted with 1 to 3 R_{X} groups.

5. The compound according to claim 4, wherein R¹ is (CH₂)ₙAr, where Ar is selected the group consisting of from biphenyl and dibenzofuranyl, where n is 1 or 2, and where Ar is optionally substituted with 1 R_{X} group; and R³ is selected from methyl, and (CH₂)ₙAr, where Ar is selected from the group consisting of phenyl, naphthyl, pyridyl, thienyl and furanyl, where n is 0, and where Ar is optionally substituted with 1 R_{X} group.

6. The compound according to claim 2, wherein the derivative is of the formula:

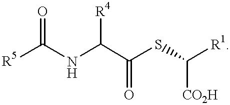

7. The compound according to claim 6, wherein R¹ is (CH₂)ₙAr, where Ar is an aryl selected from the group consisting of biphenyl and dibenzofuranyl, where n is 1 or 2, and where Ar is optionally substituted with 1 R_{X} group; and R⁴ is methyl.

8. A thiol derivative compound of the formula: 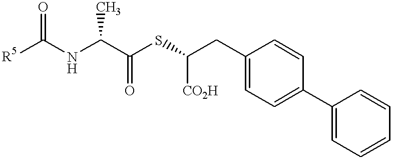
wherein:
R⁵ is selected from the group consisting of CH₃, CH₃CH₂, CH₃CH₂CH₂, CH₃(CH₂)₃, HO₂C(CH₂) ₂, H₂C═CHCH₂O, (CH₃)₂CHCH₂, (CH₃)₂CH, CH₃(CH₂)₄, HO₂CCH₂SCH₂, (E)—CH₃CH═CH, HO₂C(CH₂)₃, phenyl, PhOCH₂, PhCH₂, PhCH₂CH₂, (E)—PhCH═CH, PhCOCH₂CH₂, PhCONHCH₂,

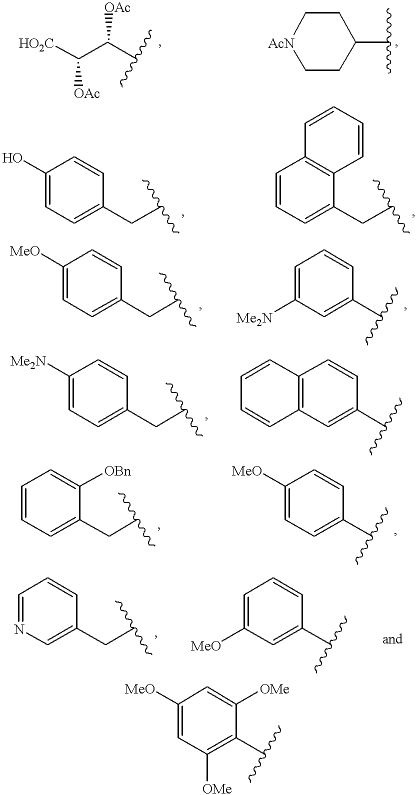

9. A thiol derivative compound of the formula: 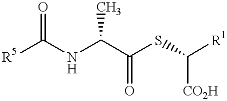
wherein R¹ and R⁵ combinations are selected from the group consisting of:

| R¹ | R⁵ |
|---|---|
| 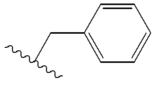 | CH₃ |
| 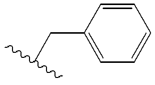 | H₂C═CHCH₂O— |
| 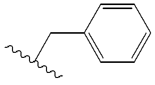 | Ph— |
| Ph— | Ph— |
| 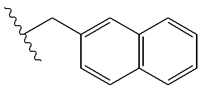 | Ph— |
| 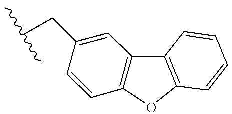 | CH₃ |
| 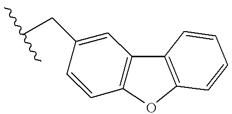 | Ph— |
| 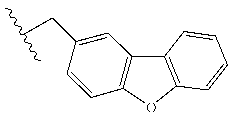 | H₂C═CHCH₂O— |
| 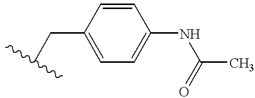 | Ph— |
| 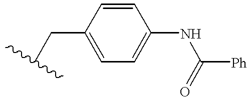 | Ph— |
| 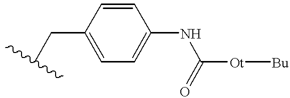 | Ph— |
| 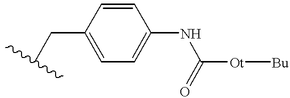 | 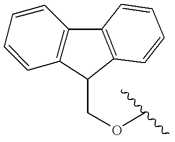 |
| 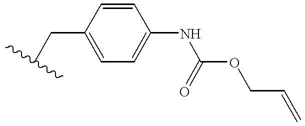 | 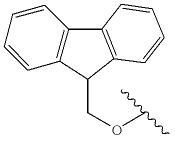 |
| 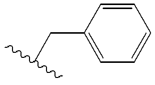 | 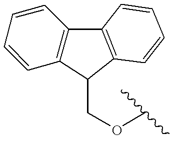 |

10. A thiol derivative compound of the formula: 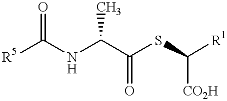
wherein R¹ and R⁵ combinations are selected from the group consisting of:

| R¹ | R⁵ |
|---|---|
| 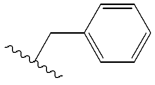 | H₂C═CHCH₂O— |
| 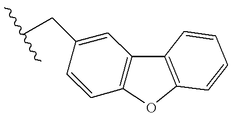 | Ph— |

11. A thiol derivative compound of the formula: 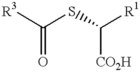
wherein R¹ and R³ combinations are selected from the group consisting of:

| R¹ | R³ |
|---|---|
| 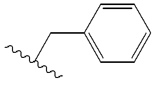 | CH₃— |
| 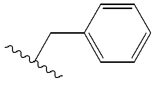 | Ph— |
| 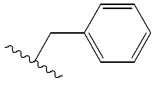 | 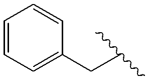 |
| 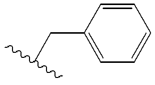 | 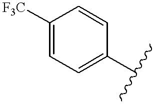 |
| 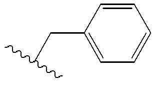 | 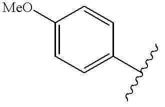 |
| 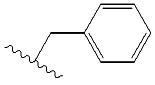 | 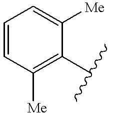 |
| 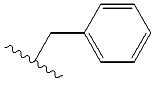 | 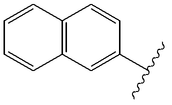 |
| 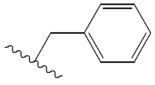 | 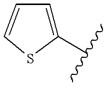 |
| 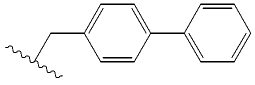 | CH₃— |
| 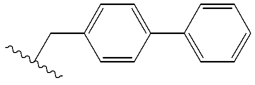 | Ph— |
| 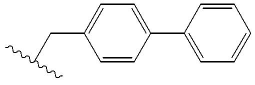 | 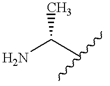 |
| 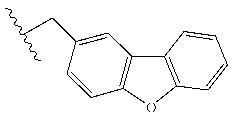 | CH₃— |
| 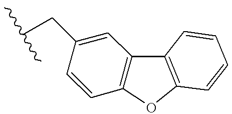 | Ph— |
| 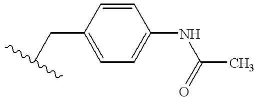 | Ph— |
| 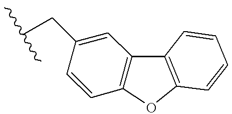 | 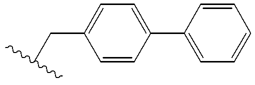 |

12. A thiol derivative compound of the formula: 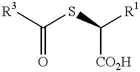
wherein R¹ and R³ combinations are selected from the group consisting of:

| R¹ | R³ |
|---|---|
| 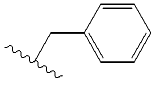 | CH₃— |
| 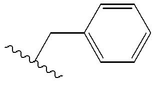 | Ph— |
| 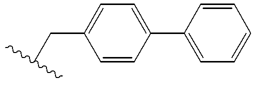 | CH₃— |
| 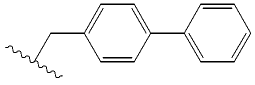 | Ph— |
| 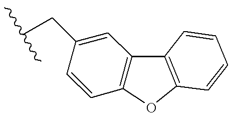 | CH₃— |
| 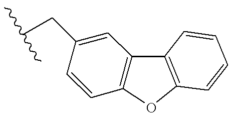 | Ph— |

13. A thiol derivative compound of the formula: 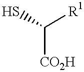
wherein R¹ is selected from the group consisting of:

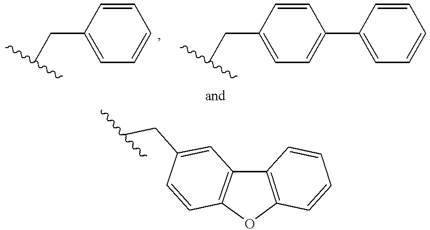

14. A thiol derivative compound of the formula: 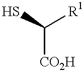
wherein R¹ is selected from the group consisting of:

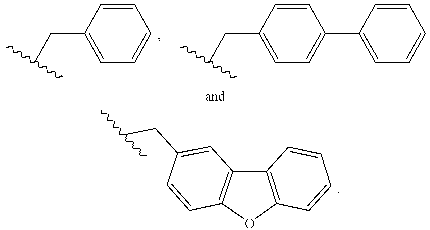

15. A pharmaceutical composition useful for treating bacterial infections in humans and animals, comprising a therapeutically effective amount of a thiol derivative, pharmaceutically acceptable salt or biolabile ester thereof, according to claim 1.

16. The composition according to claim 15, wherein the thiol derivative is selected from the group consisting of formulae Ia and Ia′:

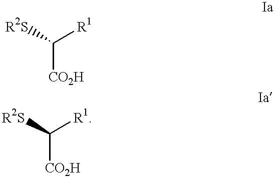

17. The composition according to claim 16, wherein the thiol derivative is of the formula Ia: 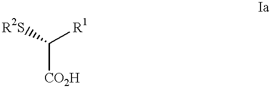
wherein R² is hydrogen.

18. The composition according to claim 16, wherein the thiol derivative is of the formula: 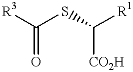
wherein:
R³ is selected from the group consisting of hydrogen; straight, branched, unsaturated or alicyclic alkyl, optionally substituted with from 1 to 3 R_{X} groups; and (CH₂)ₙAr, where Ar is an aryl selected from the group consisting of phenyl, furanyl, thienyl, pyridyl, naphthyl, biphenyl, dibenzofuranyl, dibenzothienyl, fluorenyl and fluorenonyl, where n is 0, 1, 2 or 3, and where Ar is optionally substituted with 1 to 3 R_{X} groups.

19. The composition according to claim 18, wherein R¹ is (CH₂)ₙAr, where Ar is selected from biphenyl and dibenzofuranyl, where n is 1 or 2, and where Ar is optionally substituted with 1 R_{X} group; and R³ is selected from methyl, and (CH₂)ₙAr, where Ar is selected from phenyl, naphthyl, pyridyl, thienyl and furanyl, where n is 0, and where Ar is optionally substituted with 1 R_{X} group.

20. The composition according to claim 16, wherein the thiol derivative is of the formula:

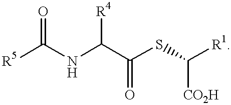

21. The composition according to claim 20, wherein R¹ is (CH₂)ₙAr, where Ar is an aryl selected from the group consisting of biphenyl and dibenzofuranyl, where n is 1 or 2, and where Ar is optionally substituted with 1 R_{X} group; and R⁴ is methyl.

22. The composition according to any one of claims 15, 16, 18 and 20, wherein the therapeutically effective amount of the compound is from about 0.1 to about 99.9 weight percent, based on 100 weight percent of the composition.

23. The composition according to claim 22, wherein the composition contains a carrier suitable for oral, topical and parenteral administration.

24. The composition according to claim 23, further comprising compounds selected from the group of β-lactam antibiotics, DHP-I inhibitors, and serine β-lactamase inhibitors.

25. The composition according to claim 24, wherein the β-lactam antibiotic is selected from the group consisting of carbapenems, penicillins and cephalosporins.

26. The composition according to claim 25, wherein the β-lactam antibiotic is a carbapenem.

27. The composition according to claim 26, wherein the carbapenem is selected from the group consisting of (1R,5S,6S,8R,2′S,4′S)-2-(2-(3-carboxyphenylcarbamoyl)pyrrolidin-4-ylthio)-6-( 1-hydroxyethyl)-1-methylcarbapenem-3-carboxylic acid and imipenem.

28. The composition according to claim 27, wherein the carbapenem is imipenem and the DHP-I inhibitor is cilastatin.

29. A method of treating bacterial infections in humans and animals, comprising administering thereto, in conjunction with a β-lactam antibiotic, a therapeutically effective amount of the composition of claim 15.

30. The method according to claim 29, wherein the thiol derivative is selected from the group consisting of formulae Ia and Ia′:

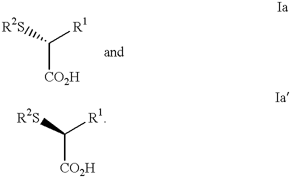

31. The method according to claim 30, wherein the thiol derivative is of the formula Ia: 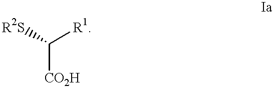
wherein R² is hydrogen.

32. The method according to claim 30, where the thiol derivative is of the formula: 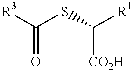
wherein:
R³ is selected from the group consisting of hydrogen; straight, branched, unsaturated or alicyclic alkyl, optionally substituted with from 1 to 3 R_{X} groups; and (CH₂)ₙAr, where Ar is an aryl selected from the group consisting of phenyl, furanyl, thienyl, pyridyl, naphthyl, biphenyl, dibenzofuranyl, dibenzothienyl, fluorenyl and fluorenonyl, where n is 0, 1, 2 or 3, and where Ar is optionally substituted with 1 to 3 R_{X} groups.

33. The method according to claim 32, where R¹ is (CH₂)ₙAr, where Ar is selected from biphenyl and dibenzofuranyl, where n is 1 or 2, and where Ar is optionally substituted with 1 R_{X} group; and R³ is selected from methyl, and (CH₂)ₙAr, where Ar is selected from phenyl, naphthyl, pyridyl, thienyl and furanyl, where n is 0, and where Ar is optionally substituted with 1 R_{X} group.

34. The method according to claim 30, where the thiol derivative is of the formula:

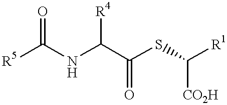

35. The method according to claim 34, wherein R¹ is (CH₂)ₙAr, where Ar is an aryl selected from the group consisting of biphenyl and dibenzofuranyl, where n is 1 or 2, and where Ar is optionally substituted with 1 R_{X} group; and R⁴ is methyl.

36. The method according to any one of claims 29, 30, 32 and 34, wherein the β-lactam antibiotic is selected from the group consisting of carbapenems, penicillins and cephalosporins.

37. The method according to claim 36, wherein the therapeutically effective amount of thiol derivative is from about 0.1 to about 99.9 weight percent, based on the total weight of the composition.

38. The method according to claim 37, wherein the β-lactam antibiotic is a carbapenem.

39. The method according to claim 38, wherein the carbapenem is selected from the group consisting of (1R,5S,6S,8R,2′S,4′S)-2-(2-(3-carboxyphenylcarbamoyl)pyrrolidin-4-ylthio)-6-(1-hydroxyethyl)-1-methylcarbapenem-3-carboxylic acid and imipenem.

40. The method according to claim 39, wherein a DHP-I inhibitor is co-administered with imipenem.

41. The method according to claim 40, wherein the DHP-I inhibitor is cilastatin.
